(19) United States
(12) Patent Application Publication
(10) Pub. No.: US 2005/0261497 A1
(43) Pub. Date: Nov. 24, 2005

(54) Catalytic asymmetric hetero diels-alder reaction of a heteroaromatic C-nitroso dienophile: a novel method for synthesis of chiral non-racemic amino alcohols

(76) Inventors: Yuhei Yamamoto, Okazaki (JP); Hisashi Yamamoto, Chicago, IL (US)

Correspondence Address: BRINKS HOFER GILSON & LIONE, P.O. BOX 10395, CHICAGO, IL, 60610, US

(21) Appl. No.: 11/027,551

(22) Filed: Dec. 30, 2004

Related U.S. Application Data
(60) Provisional application No. 60/534,025, filed on Jan. 2, 2004.

(51) Int. Cl. C07D 491/04

(52) U.S. Cl. 544/63

(57) ABSTRACT

The present invention is directed to a catalytic asymmetric C-nitroso Diels-Alder reaction.

### RELATED APPLICATIONS

This application claims priority to U.S. provisional application Ser. No. 60/534,025 filed Jan. 2, 2004. The disclosure of the priority application is incorporated by reference herein in its entirety.

### BACKGROUND OF THE INVENTION

Dihydro-1,2-oxazine derivatives are an important class of compounds, which have been used to access a large variety of nitrogenous molecules. See Hall, A.; Bailey, P. D.; Rees, D. C.; Rosair, G. M.; Wightman, R. H. J. Chem. Soc., Perkin Trans. 2000, 1, 329-343. For example, dihydro-1,2-oxazines can easily be converted to amino alcohols, which, with their dual functionality, play an important role in a variety of industrial processes and are also important components in numerous household goods and personal care products. Additionally, dihydro-1,2-oxazines have been converted to biologically active amino alcohols, such as aminocyclitols, inosamines, and conduramines. See Streith, J.; Defoin, A. Synthesis 1994, 1107-1117. Dihydro-1,2-oxazines have also been transformed into a wide variety of biologically active natural products. These include naturally occurring pyrrolidine and piperdine alkaloids, as well as indolizidine and pyrrolizidine alkaloids. One noteworthy alkaloid, which has been derived from a dihydro-1,2-oxazine, is epibatidine. In fact, this compound, which is isolated from the skin of the Ecuadorean frog Epipedobates tricolor, has been shown to have potent analgesic effects while being devoid of opiate activity. Cheng, J.; Zhang, C.; Stevens, E. D.; Izenwasser, S.; Wade, D.; Chen, S.; Paul, D.; Trudell, M. L. J. Med. Chem. 2002, 45, 3041-3047.

To date, there has been a significant amount of research focusing on Diels-Alder reactions utilizing C-nitroso dienophiles. For example, numerous diastereoselective variations of this reaction, utilizing chiral auxiliaries, have been reported. See Vogt, P. F.; Millar, M. J. Tetrahedron 1998, 54, 1317-1348. Unfortunately, these reactions are typically costly, due to a required stoichiometric quantity of chiral auxiliary. Furthermore, reactions employing chiral auxiliaries are also complicated by the additional step needed to remove this auxiliary. Additionally, catalytic asymmetric reactions are usually much more amendable to large scale syntheses, which is important for the production of pharmaceutical compounds. Thus, a catalytic asymmetric Diels-Alder reaction, utilizing C-nitroso dienophiles, would be ideal. However, prior to the work disclosed herein, attempts to create an asymmetric catalytic variation of this reaction have been unsuccessful. See Lightfoot, A. P., Pritchard, R. G., Wan, H., Warren, J. D., and Whiting, A., Chem. Commun., 2002, 2072-2073; Ding, X., Ukaji, Y., Fujinami, S., Inomata, K., Chemistry Letters, 32, No. 7 (2003).

### BRIEF SUMMARY OF THE INVENTION

The present invention is directed to a process of enantioselective chemical synthesis, consisting of reacting a C-nitroso dienophile and a 1,3-diene in the presence of a catalytic amount of an asymmetric bidentate ligand and a metal, to produce an enantiomerically enriched cycloadduct.

Not only does this catalytic asymmetric C-nitroso Diels-Alder reaction generate two asymmetric centers, in a one-step catalytic process, but it also provides access to dihydro-1,2-oxazines, which can be further converted to a variety of nitrogenous biologically active compounds, including amino alcohols.

### DETAILED DESCRIPTION OF THE INVENTION

The Diels-Alder Reaction

The system described herein relies on the formation of indirect conjugates in which two molecules are joined via a Diels-Alder adduct. The Diels-Alder reaction was first described in 1928 and provides a convenient and highly stereospecific route to a 6-membered ring. The reactants are a diene and a dienophile. These reactants approach each other on approximately parallel planes and react to form a 6-membered ring (hereinafter a “cycloadduct”):

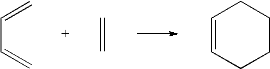

(Diels & Alder, Justus Liebigs Ann. Chem., 1928, 460, 98; Numerous references have reviewed this chemistry including, Wassermann, “Diels-Alder Reactions;” Elsevier, Amsterdam, 1965; Sauer et al., Angew. Chem. Int. Ed. Engl. 1980, 19, 779; Hoffmann, Angew. Chem. Int. Ed. Engl. 1984, 23, 1).

General

The present invention is directed to a catalytic asymmetric C-nitroso Diels-Alder reaction. This methodology generally comprises reacting a C-nitroso dienophile and a 1,3-diene, in the presence of a catalytic amount of an asymmetric bidentate ligand and a metal, to provide an enantiomerically enriched dihydro-1,2-oxazine cycloadduct with two asymmetric centers.

In one embodiment, the Diels-Alder reaction can be represented as follows: 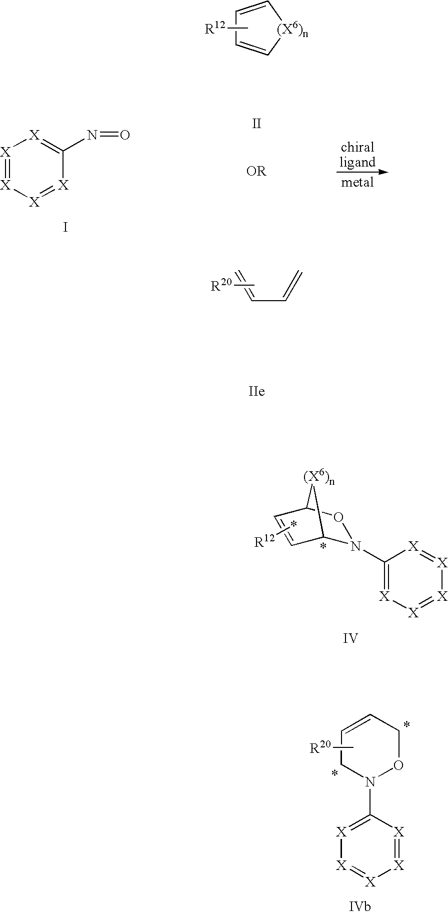
where, compound I represents one embodiment of a C-nitroso dienophile, in this case an aromatic C-nitroso dienophile. Compounds II and lie correspond to two embodiments of a 1,3-diene, a cyclic diene and a acyclic diene respectively. Finally, compounds IV and IVb embody two dihydro-1,2-oxazine cycloadducts, derived via the Diels-Alder reaction of a six-membered C-nitroso dienophile (I) and cyclic and acyclic dienes, respectively.
Abbreviations and Definitions

When describing the compounds, compositions, methods and processes of this invention, the following terms have the following meanings, unless otherwise indicated.

“Alkyl” by itself or as part of another substituent refers to a hydrocarbon group which may be linear, cyclic, or branched or a combination thereof having from 1 to 10 carbon atoms (preferably 1 to 8 carbon atoms). Examples of alkyl groups include methyl, ethyl, n-propyl, isopropyl, n-butyl, t-butyl, isobutyl, sec-butyl, cyclohexyl, cyclopentyl, (cyclohexyl)methyl, cyclopropylmethyl and the like.

“Cycloalkyl” refers to hydrocarbon rings having from 3 to 12 carbon atoms and being fully saturated or having no more than one double bond between ring vertices (preferably 5 to 6 carbon atoms). Examples of cycloalkyl include cyclopropyl, cyclopentyl, cycloyhexyl and the like. “Cycloalkyl” is also meant to refer to bicyclic and polycyclic hydrocarbon rings such as, for example, bicyclo[2.2.1]heptane, bicyclo[2.2.2]octane, and the like.

“Alkoxy” refers to those alkyl groups, having from 1 to 10 carbon atoms, attached to the remainder of the molecule via an oxygen atom. Alkoxy groups with 1-8 carbon atoms are preferred. The alkyl portion of an alkoxy may be linear, cyclic, or branched or a combination thereof. Examples of alkoxy groups include methoxy, ethoxy, isopropoxy, butoxy, cyclopentyloxy, and the like. An alkoxy group can also be represented by the following formula: —OR′, where R′ is the “alkyl portion” of an alkoxy group.

“Alkylamino” refers to those alkyl groups, having from 1 to 10 carbon atoms, attached to the remainder of the molecule via a nitrogen atom. Alkylamino groups with 1-8 carbon atoms are preferred. The alkyl portion of an alkylamino may be linear, cyclic, or branched or a combination thereof. Examples of alkylamino groups include methyl amine, ethyl amine, isopropyl amine, butyl amine, dimethyl amine, methyl, isopropyl amine and the like. An alkylamino group can also be represented by the following formulae: —NR′— or —N′R″, or —NHR′, where R′ and R″ are the “alkyl portion” of an alkylamino group.

“Alkylthio” refers to those alkyl groups, having from 1 to 10 carbon atoms, attached to the remainder of the molecule via a sulfur atom. Alkylthio groups with 1-8 carbon atoms are preferred. The alkyl portion of an alkylthio may be which may be linear, cyclic, or branched or a combination thereof. Examples of alkylthio groups include methyl sulfide, ethyl sulfide, isopropyl sulfide, butyl sulfide and the like. An alkylthio group can be represented by the formula: —SR, where R is the “alkyl portion” of an alkylthio group.

“Aryl” refers to an aromatic hydrocarbon group having a single ring or multiple rings which are fused together or linked covalently with 5 to 14 carbon atoms (preferably 5 to 10 carbon atoms). Examples of aryl groups include phenyl, naphthalene-1-yl, naphthalene-2-yl, biphenyl, anthracene and the like.

“Arylalkyl” refers to an aryl group, where a free valence resides on an alkyl side chain. Such groups may have single or multiple substituents on either the aryl ring or on the alkyl side chain. Examples include benzyl, phenylethyl, styryl, 2-(4-methylphenyl)ethyl, and 2-phenylpropyl.

“Halo” or “halogen”, by itself or as part of a substituent refers to a chlorine, bromine, iodine, or fluorine atom. Additionally, terms such as “Haloalkyl” refer to a monohaloalkyl or polyhaloalkyl group, most typically substituted with from 1-3 halogen atoms. Examples include 1-chloroethyl, 3-bromopropyl, trifluoromethyl and the like.

“Heterocyclyl” refers to a saturated or unsaturated non-aromatic group containing at least one heteroatom and having 3 to 10 members (preferably 3 to 7 carbon atoms). “Heteroaryl group” refers to an aromatic group containing at least one heteroatom and having 3 to 10 members (preferably 3 to 7 carbon atoms). Each heterocyclyl and heteroaryl can be attached at any available ring carbon or heteroatom. Each heterocyclyl may have one or more rings. When multiple rings are present in a heterocyclyl, they can be fused together or linked covalently. Each heteroaryl may have one or more rings. When multiple rings are present in a heteroaryl, they can be fused. Each heterocyclyl and hetroaryl can be fused to a cyclyl, heterocyclyl, heteroaryl, or aryl group. Each heterocyclyl and heteroaryl must contain at least one heteroatom (typically 1 to 5 heteroatoms) selected from nitrogen, oxygen or sulfur. Preferably, these groups contain 0-3 nitrogen atoms and 0-1 oxygen atoms. Examples of saturated and unsaturated heterocyclyl groups include pyrrolidine, imidazolidine, pyrazolidine, piperidine, 1,4-dioxane, morpholine, piperazine, 3-pyrroline and the like. Examples of heteroaryl groups include pyrrole, imidazole, oxazole, furan, triazole, tetrazole, oxadiazole, pyrazole, isoxazole, pyridine, pyrazine, pyridazine, pyrimidine, triazine, indole, benzofuran, benzimidazole, benzopyrazole, quinoline, isoquinoline, quinazoline, quinoxaline and the like. Heterocyclyl and heteroaryl groups can be unsubstituted or substituted. For substituted groups, the substitution may be on a carbon or heteroatom. For example, when the substitution is ═O, the resulting group may have either a carbonyl (—C(O)—) or a N-oxide (—N(O)—).

“Dihydro-1,2-oxazine cycloadduct,” “dihyro-1,2-oxazine,” or “cycloadduct,” refers to the initial product resulting from the reaction disclosed herein. For example, when a C-nitroso dienophile, such as I, is employed in combination with a diene, such as II, then the dihydro-1,2-oxazine is of the formula (IV):

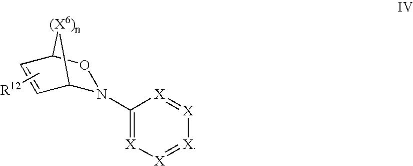

In another example, when a C-nitroso dienophile, such as Ia, is employed in combination with a diene such as 1, then the dihydro-1,2-oxazine cycloadduct is of the formula (IVa):

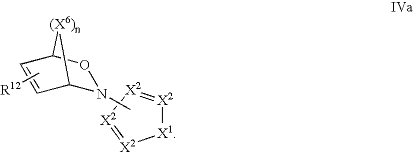

All of the above terms (e.g., “alkyl,” “aryl,” “heteroaryl” etc.) include both substituted and unsubstituted forms of the indicated groups. These groups may be substituted 1 to 10 times. Examples of substituents include alkyl, cycloalkyl, alkoxy, alkylamino, alkylthio, aryl, arylalkyl, heterocyclyl, heteroaryl, halogen, silyloxy, carboxylic acid, ester, alkene, azide, amine, hydroxyl, imine, ketone, thiole, amide, silyl, nitrile, sulfoxide, sulfone, sulfonamide and nitroso.

“Transition metal” or “metal” refers to a chemical element that either has incompletely filled d subshells or readily give rise to cations that have incompletely filled d subshells. The elements in the periodic table from and including IIIB to IIB belong to the transition metals. The metal may be present as a pure metal or metal ion or may be present in an association with one or more ligands. Examples of a metal include CuPF₆(MeCN)₄, Cu(OTf)₂, Cu(SbF₆)₂, [CuOTf] benzene, CuSbF₆, AgSbF₆ and Pd(BF⁴).

“Lewis acid” refers to a molecular entity (and the corresponding chemical species) that is an electron-pair acceptor and therefore able to react with a Lewis base to form a Lewis adduct, by sharing the electron pair furnished by the Lewis base. For example:
Me₃B (Lewis acid)+:NH₃ (Lewis base)→Me₃B⁻—N⁺H₃ (Lewis adduct)

Examples of Lewis acids include H⁺, Li⁺, Na⁺, Zn²⁺, Pd²⁺, Ag⁺, and Cu⁺. A Lewis base is a molecular entity that is an electron-pair donor.

“Transition metal Lewis acid” refers to a molecular entity (and the corresponding chemical species) of groups 3-10 of the periodic table that is an electron-pair acceptor. Examples of Transition metal Lewis acid include Sc³⁺, Ti⁴⁺, Co²⁺, Fe³⁺, Zn²⁺, Pd²⁺, Ag⁺, and Cu⁺.

“1,3-Diene” refers to a molecule containing at least one pair of conjugated π-bonds. The individual π-bonds of the diene moiety may be between any two atoms selected from the group consisting of C, N, O, S, and P. The conjugated π-bonds of the diene must be capable of adopting the so-called s-cis conformation.

“Dienophile” refers to a molecule containing at least one reactive π-bond. The reactive π-bond of the dienophile can be chosen from the following formulae: R¹N═O, R¹N═S, R¹N═N, R¹N═CR²R³, R¹R²C═O, R¹R²C═N, R¹R²C═S, O═O, S═S, and R¹R²C═CR³R⁴.

“C-nitroso dienophile” or “nitroso dienophile” refer to a molecule containing a reactive π-bond, which is located between a nitrogen atom and an oxygen atom.

“Asymmetric” refers to a molecule lacking all elements of symmetry. For example, the following carbon center is asymmetric:

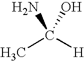

“Chiral” refers to a molecule or conformation which is not superimposable with its mirror image partner. The term “achiral” refers to molecule or conformation which is superimposable with its mirror image partner.

“Asymmetric bidentate ligand” refers to a molecule lacking all elements of symmetry in which there are two Lewis base or electron pair donor atoms present, to act as ligands.

“Enantiomer” refers to one of a pair of molecular species that are mirror images of each other and not superposable.

“Enantiomerically enriched” refers to a mixture of enantiomers, in which one of the enantiomers has been selectively created in preference over the other enantiomer. Thus an “enantiomerically enriched” product will have an enantiomeric excess (i.e., % ee), in which one enantiomer is present in a larger amount than the other. To put it another way, “enantiomerically enriched” refers to having an enantiomer excess of more than 0 but less than 100%. “Enantiomeric excess” is equal to 100 times the mole fraction of the major enantiomer minus the more fraction of the minor enantiomer. In a mixture of a pure enantiomer (R or S) and a racemate, ee is the percent excess of the enantiomer over the racemate.

“Enantioselective” refers to a process which favors production of one of the two possible enantiomers of a reaction product. For example, a chemical reaction would be enantioselective if it produces the two enantiomers of a chiral product in unequal amounts. Such a reaction is said to exhibit enantioselectivity.

“Complex” refers to a coordination compound formed by the union of one or more electronically rich molecules or atoms capable of independent existence with one or more electronically poor molecules or atoms, which is also capable of independent existence.

“Ligand” refers to the molecules or ions that surround the metal in a complex and serve as Lewis bases (i.e., electron pair donors).

“Chiral ligand” refers to a molecule or ion that surrounds a metal in a metal ion complex as a Lewis base, where the molecule is one which is not superimposable with its mirror image partner.

“Catalytic amount” refers to a substoichiometric amount of the catalyst relative to a reactant.

“Catalysis” or “catalyzed” refer to a process in which a relatively small amount of a foreign material increases the rate of a chemical reaction and is not itself consumed in the reaction.

“Chiral catalyst” refers to a molecule or conformation, which is not superimposable with its mirror image partner and that increases the rate of a chemical reaction without itself being consumed. In an asymmetric catalytic reaction, the chiral catalyst will serve to catalyze the reaction, while also providing enantioselectivity.

“Hetero Diels-Alder reaction” refers to a [4+2] cycloaddition between a dienophile and diene in which one or more atoms of the diene or dienophile is a heteratom. Thus the product of a hetero Diels-Alder reaction is a heterocyclyl group.

“Heteroatom” refers to an atom other than carbon. Examples include nitrogen, oxygen, sulfur, phosphorus and the like.

“O-silyl” refers to an oxygen atom which is substituted with a silyl group and another group. Examples of O-silyl groups include O-trimethylsilyl (abbreviated to be—OTMS), O-triethylsilyl, O-triphenylsilyl, O-di-tert-butyl-methyl-silyl (abbreviated to be—OTBS) and the like.

“Inert atmosphere” refers to reaction conditions in which the mixture is covered with a layer of inert gas such as nitrogen or argon.

“Substituted” refers to a moiety that has at least one, preferably 1 to 3 substituent(s). Suitable substituents include alkyl, cycloalkyl, alkoxy, alkylamino, alkylthio, aryl, arylalkyl, heterocyclyl, heteroaryl, halogen, silyloxy, carboxylic acid, ester, alkene, azide, amine, hydroxyl, imine, ketone, thiole, amide, silyl, nitrile, sulfoxide, sulfone, sulfonamide and nitroso. These substituents can optionally be further substituted with 1 to 10 substituents. Examples of substituted substituents include alkylamino, dialkylamino, alkylaryl, arylalkyl, 2-methyl-pyridine, 3-chloropropane, and the like.

C-nitroso Dienophile

In the present invention, the C-nitroso dienophile can consist of an aromatic ring with an attached C-nitroso substituent represented by formula I, where each X (X group or X substituent) is independently selected from the group consisting of —CR¹— or —N—. In one preferred embodiment, at least one X is —N—. In another preferred embodiment, at least two X groups are —N—.

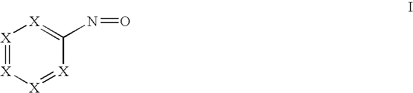

Each R¹ group of compound I can be independently selected from the group consisting of hydrogen, alkyl, cycloalkyl, alkoxy, alkylamino, alkylthio, halogen, heterocyclyl, aryl, arylalkyl, heteroaryl, and O-silyl. In one embodiment, each R¹ is independently selected from the group consisting of hydrogen, alkyl, cycloalkyl, and heterocyclyl. In another embodiment, each R¹ is independently selected from the group consisting of alkoxy, alkylamino, alkylthio, and halogen. In an additional embodiment, each R¹ is independently selected from the group consisting of aryl, heteroaryl, arylalkyl, and O-silyl. With regard to C-nitroso dienophile I, R¹ preferably represents hydrogen, alkyl, cycloalkyl, aryl, arylalkyl, halogen, and O-silyl.

Choosing certain groups for X can affect the identity of C-nitroso dienophile I, as well as the number of R¹ substituents. If one X group is nitrogen (—N—) and the remaining X groups are carbon, then the C-nitroso dienophile is a C-nitroso substituted pyridine with four R¹ groups. Moreover, if two of the X groups are nitrogens (—N—), while the remainder of X groups are carbon, then dienophile I could be a C-nitroso substituted pyrimidine, pyrazine, or pyridazine, with three R¹ groups.

The size of the C-nitroso dienophile ring can vary. For example, the C-nitroso dienophile can be a 6-membered ring, as in compound I above. Alternatively the C-nitroso dienophile can be a compound of the formula (Ia): 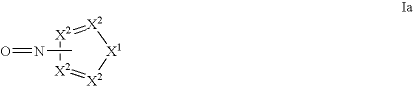
where, the dienophile is a 5-membered ring. The X¹ substituent can be selected from the group consisting of —NR²—, —O—, and —S—. In one embodiment, X¹ is —NR²—. In another embodiment X¹ is selected from the group consisting of —O— and —S—. However, X¹ is preferably nitrogen (—NR²—). The X² substituents of Ia are independently selected from the group consisting of —CR³— or —N—.

With respect to compound Ia, groups R² and R³ are independently selected from the group consisting of hydrogen, alkyl, cycloalkyl, alkoxy, alkylamino, alkylthio, halogen, heterocyclyl, aryl, heteroaryl, arylalkyl, and O-silyl. In one embodiment, R² and R³ are independently selected from the group consisting of hydrogen, alkyl, cycloalkyl, and heterocyclyl. In another embodiment, R² and R³ are independently selected from the group consisting of alkoxy, alkylamino, alkylthio, and halogen. In an additional embodiment, R² and R³ are independently selected from the group consisting of aryl, heteroaryl, arylalkyl, and O-silyl. However, R² and R³ are preferably hydrogen, alkyl, cycloalkyl, aryl, arylalkyl, halogen and O-silyl.

Choosing certain groups for X¹ and X² can affect the identity of C-nitroso dienophile Ia. For example if X¹ is nitrogen (—NR²—) and each X² is carbon (—CR³—), the C-nitroso dienophile (Ia) would be a C-nitroso substituted 1H-pyrrole with one R² group and four R³ groups. If X¹ is nitrogen (—NR²—), and one X² is nitrogen (—N—), then dienophile Ia would be a 1H-pyrazole or a 1H-imidazole.

In another embodiment, the Diels-Alder reaction is performed when the dienophile is a compound of formula (Ib): 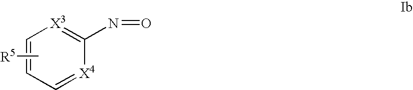
where, X³ and X⁴ are independently selected from the group consisting of —CR⁴— or —N—. Thus one could choose X³ and X⁴ such that: one of these groups is nitrogen (—N—) and one is carbon (—CR⁴—), in which case Ib is 2-nitrosopyridine; both groups are nitrogen atoms (—N—), in which case Ib is a 2-nitrosopyrimidine; or both groups are carbons (—CR⁴—), in which case Ib is a 1-nitrosobenzene. Preferably, at least one of X³ and X⁴ is a nitrogen atom (—N—).

The R⁴ substituents of Ib are each independently selected from the group consisting of hydrogen, alkyl, cycloalkyl, alkoxy, alkylamino, alkylthio, O-silyl, aryl, arylalkyl, heteroaryl, heterocyclyl and halogen. In one embodiment, the reaction is performed when each R⁴ is independently selected from the group consisting of hydrogen, alkyl, and cycloalkyl. In another embodiment, each R⁴ is independently selected from the group consisting of alkoxy, alkylamino, alkylthio, and O-silyl. In an additional embodiment, each R⁴ is independently selected from the group consisting of aryl, arylalkyl, heteroaryl, heterocyclyl and halogen. However, R¹ is preferably alkyl, cycloalkyl, aryl, arylalkyl, halogen, or O-silyl.

The R⁵ group of Ib represents zero to three substituents, each of which is independently selected from the group consisting of alkyl, cycloalkyl, alkoxy, alkylamino, alkylthio, halogen, heterocyclyl, aryl, heteroaryl, arylalkyl, and O-silyl. In one embodiment, R⁵ is independently selected from the group consisting of hydrogen, alkyl, cycloalkyl, and heterocyclyl. In another embodiment, R⁵ is independently selected from the group consisting of alkoxy, alkylamino, alkylthio, and O-silyl. In an additional embodiment, R⁵ is independently selected from the group consisting of aryl, heteroaryl, arylalkyl and halogen. However, R⁵ is preferably hydrogen, alkyl, cycloalkyl, aryl and O-silyl.

The Diels-Alder reaction can be performed when the C-nitroso dienophile (I) is a compound of formula (Ic): 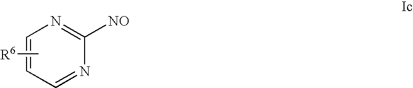
where, R⁶ represents zero to three substituents, each of which can be independently selected from the group consisting of alkyl, cycloalkyl, alkoxy, alkylamino, alkylthio, halogen, heterocyclyl, aryl, heteroaryl, arylalkyl, and O-silyl. In one embodiment, R⁶ is independently selected from the group consisting of hydrogen, alkyl, cycloalkyl, and heterocyclyl. In another embodiment, R⁶ is independently selected from the group consisting of alkoxy, alkylamino, alkylthio, and O-silyl. In an additional embodiment, R⁶ is independently selected from the group consisting of aryl, heteroaryl, arylalkyl and halogen. However, R⁶ is preferably alkyl, cycloalkyl, aryl, arylalkyl, halogen and O-silyl.

The Diels-Alder reaction can also be performed when the dienophile is a compound of formula (Id): 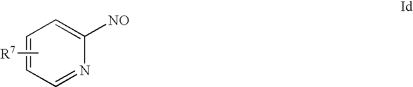
where, R⁷ represents zero to four substituents, each of which can be independently selected from the group consisting of alkyl, cycloalkyl, alkoxy, alkylamino, alkylthio, O-silyl, aryl, arylalkyl, heteroaryl, heterocyclyl and halogen. In one embodiment, R⁷ is independently selected from the group consisting of alkyl and cycloalkyl. In another embodiment, R⁷ is independently selected from the group consisting of alkoxy, alkylamino, alkylthio, and O-silyl. In an additional embodiment, R⁷ is independently selected from the group consisting of aryl, heteroaryl, heterocyclyl and halogen. However, the R⁷ is preferably selected from the group consisting of aryl, heteroaryl, arylalkyl, heterocyclyl, halogen, and O-silyl.

In another embodiment, the Diels-Alder reaction is performed when the C-nitroso dienophile (I) is a compound of formula (Ie): 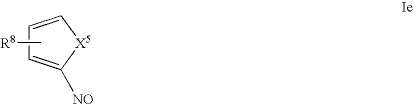
where, X⁵ is selected from the group consisting of —NR¹—, —O—, or —S—. In one embodiment X⁵ is —NR¹—. In another embodiment X⁵ is selected from the group consisting of —O— or —S—. The X⁵ group is preferably —NR¹—.

The R⁸ group Ie, represents zero to three substituents, each of which is independently selected from the group consisting of alkyl, cycloalkyl, alkoxy, alkylamino, alkylthio, halogen, heterocyclyl, aryl, heteroaryl, arylalkyl, and O-silyl. In one embodiment, R⁸ is independently selected from the group consisting of hydrogen, alkyl, and cycloalkyl. In another embodiment, R⁸ is independently selected from the group consisting of alkoxy, alkylamino, alkyl sulfide, and O-silyl. In an additional embodiment, R⁸ is independently selected from the group consisting of aryl, heteroaryl, arylalkyl, heterocyclyl and halogen. However, R⁵ is preferably selected from the group consisting of hydrogen, alkyl, cycloalkyl, aryl, arylalkyl, halogen and O-silyl.

The R⁹ group of Ie, can be independently selected from the group consisting of hydrogen, alkyl, cycloalkyl, alkoxy, alkylamino, alkylthio, halogen, heterocyclyl, aryl, heteroaryl, arylalkyl, and O-silyl. In one embodiment, R⁹ is independently selected from the group consisting of hydrogen, alkyl, and cycloalkyl. In another embodiment, R⁹ of Ie is independently selected from the group consisting of alkoxy, alkylamino, alkyl sulfide, and O-silyl. In an additional embodiment, R⁹ is independently selected from the group consisting of aryl, arylalkyl, heteroaryl, heterocyclyl and halogen. However, R⁹ is preferably selected from the group consisting of hydrogen, alkyl, cycloalkyl, aryl, arylalkyl and O-silyl.

In a preferred embodiment, the Diels-Alder reaction is performed when the C-nitroso dienophile (I) is selected from the group consisting of 2-nitrosopyridine, 3-methyl-2-nitrosopyridine, 2-nitrosopyrimidine, 2-methyl-6-nitrosopyridine, 2-ethyl-6-nitrosopyridine, or 2-isopropyl-6-nitrosopyridine.

In another preferred embodiment, the Diels-Alder reaction is performed when the C-nitroso dienophile is a compound of formula (If): 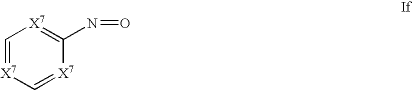
where, each X⁷ is selected from the group consisting of —CR²⁷— or —N—; and at least one X⁷ is —N—. Furthermore, the R²⁷ substituent is independently selected from the group consisting of hydrogen, alkyl, cycloalkyl, alkoxy, halogen, heterocyclyl, aryl, heteroaryl, arylalkyl, and O-silyl.
Diene (II) and (IIa)

In the present invention the diene can be either cyclic (II) or acyclic (IIa).

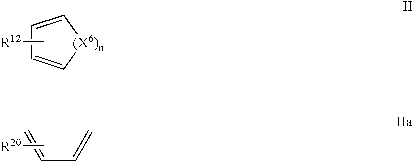

With regard to cyclic diene II, R¹² represents zero to four substituents, each of which is independently selected from the group consisting of alkyl, cycloalkyl, alkoxy, alkylamino, alkylthio, aryl, arylalkyl, heterocyclyl, heteroaryl, halogen, silyloxy, carboxylic acid, ester, alkene, azide, amine, hydroxyl, imine, ketone, thiole, amide, silyl, nitrile, sulfoxide, sulfone, sulfonamide and nitroso. When two R¹² or R²⁰ groups are present and are adjacent to each other, they may form a ring, together with the atoms to which they are attached. For example, two adjacent R¹² or R²⁰ substituents may form a cycloalkyl ring or where two adjacent R¹² or R²⁰ substituents are alkoxy they may form a heterocyclic ring.

As discussed below, the value of n will alter the ring size of cyclic diene II. In one embodiment, R¹² is independently selected from the group consisting of alkyl, cycloalkyl, and heterocyclyl. In another embodiment, R¹² is independently selected from the group consisting of alkoxy, alkylamino, alkylthio, and halogen. In an additional embodiment, R¹² is independently selected from the group consisting of aryl, heteroaryl, arylalkyl, and O-silyl. Preferably R¹² is selected from the group consisting of alkyl, cycloalkyl, aryl, arylalkyl, halogen, and O-silyl.

The X⁶ moiety of diene II can be independently selected from the group consisting of —CR⁹R¹⁰—, —NR¹¹—, —O—, and —S—. Furthermore n is 1, 2, 3, or 4. As the value of n increases, the size of the ring increases. For example, if n is 2, diene II is a ring with six members. If n is 3, then diene II is a ring with seven members and so on. It should also be noted that if the value of n is more than one, there will be multiple X⁵ ring members. If there are multiple X⁵ ring members within diene II, it is important to realize that each X⁵ substituent is independently selected from the group previously described. Thus if n is 2, there will be two X⁵ ring members, each of which can be independently selected from the group consisting of —CR¹R^{1′}—, —NR¹—, O—, and —S—. In one embodiment, the Diels-Alder reaction is performed when n of diene II is 1 or 2. In another embodiment n of diene II is 3 or 4. In a preferred embodiment, n is 1, 2, or 3.

In one embodiment, X⁶ is independently selected from the group consisting of —CR⁹R¹⁰— and —NR¹¹—. In another embodiment, X⁵ is independently selected from the group consisting of —O— and —S—.

The R⁹, R¹¹, and R¹⁰ substituents of diene II are independently selected from the group consisting of hydrogen, alkyl, cycloalkyl, alkoxy, alkylamino, alkylthio, halogen, heterocyclyl, aryl, heteroaryl, arylalkyl, and O-silyl. In one embodiment, R⁹, R¹¹, and R¹⁰ are each independently selected from the group consisting of hydrogen, alkyl, cycloalkyl, and heterocyclyl. In another embodiment, R⁹, R¹¹, and R¹⁰ are each independently selected from the group consisting of alkoxy, alkylamino, alkylthio, and halogen. In an additional embodiment, R⁹, R¹¹, and R¹⁰ are each independently selected from the group consisting of aryl, heteroaryl, arylalkyl, and O-silyl. Preferably, R⁹, R¹¹, and R¹⁰ are each independently selected from the group consisting of hydrogen, alkyl, cycloalkyl, aryl, arylalkyl, halogen, and O-silyl.

The Diels-Alder reaction, described herein, can be performed with a variety of dienes. For example, the cyclic diene can be selected from the following formulae (IIa, IIb, IIc, and IId): 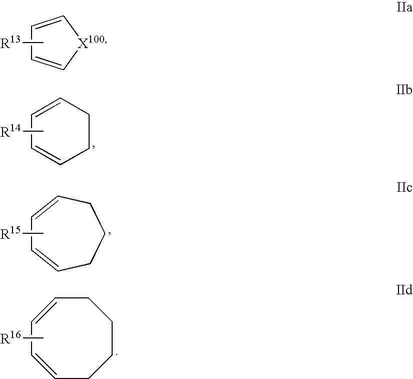
where, R¹³ represents zero to four substituents in IIa; R¹⁴ represents zero to eight substituents in IIb; R¹⁵ represents zero to ten substituents in IIc; and R¹⁶ represents zero to twelve substituents in IId. R¹³, R¹⁴, R¹⁵, and R¹⁶ are each independently selected from the group consisting of alkyl, cycloalkyl, alkoxy, alkylamino, alkylthio, halogen, heterocyclyl, aryl, arylalkyl, heteroaryl, and O-silyl. In one embodiment, R¹³, R¹⁴, R¹⁵, and R¹⁶ are each independently selected from the group consisting of alkyl, cycloalkyl, and heterocyclyl. In another embodiment, R¹³, R¹⁴, R¹⁵, and R¹⁶ are each independently selected from the group consisting of alkoxy, alkylamino, alkylthio, and halogen. In an additional embodiment, R¹³, R¹⁴, R¹⁵, and R¹⁶ are each independently selected from the group consisting of aryl, heteroaryl, arylalkyl, and O-silyl. Preferably, R¹³, R¹⁴, R¹⁵, and R¹⁶ are each independently selected from the group consisting of alkyl, cycloalkyl, aryl, arylalkyl, halogen, and O-silyl. As described above for R¹² and R²⁰, when two of R¹³, R⁴, R¹⁵ or R¹⁶ are present on the same diene and are adjacent to each other, they may form a ring.

The X¹⁰⁰ substituent of diene IIa is selected from the group consisting of —CR¹⁷R¹⁸—, —NR¹⁹—, —O—, and —S—. In one embodiment, X¹⁰⁰ is selected from the group consisting of —CR¹⁷R¹⁸— and —NR¹⁹—. In another embodiment, X¹⁰⁰ is selected from the group consisting of —O— and —S—.

The R¹⁷, R¹⁸, and R¹⁹ substituents of —CR¹⁷R¹⁸— and —NR¹⁹— are independently selected from the group consisting of hydrogen, alkyl, cycloalkyl, alkoxy, alkylamino, alkylthio, halogen, heterocyclyl, aryl, heteroaryl, arylalkyl, and O-silyl;

As previously mentioned, this catalytic asymmetric C-nitroso Diels-Alder reaction can be performed when the diene is a acyclic diene, such as compound IIe.

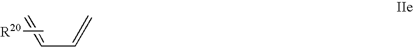

The R²⁰ substituent of IIe represents zero to six substituents, each of which can be independently selected from the group consisting of alkyl, cycloalkyl, alkoxy, alkylamino, alkylthio, halogen, heterocyclyl, aryl, heteroaryl, arylalkyl, and O-silyl. In one embodiment, each R²⁰ is independently selected from the group consisting of alkyl, cycloalkyl, and heterocyclyl. In another embodiment, R²⁰ is independently selected from the group consisting of alkoxy, alkylamino, alkylthio, and halogen. In an additional embodiment, each R²⁰ is independently selected from the group consisting of aryl, heteroaryl, arylalkyl, and O-silyl. Preferably, each R²⁰ substituent is independently selected from the group consisting of alkyl, cycloalkyl, aryl, arylalkyl, O-silyl, and halogen.

The catalytic asymmetric C-nitroso Diels-Alder reaction, disclosed herein, can be performed with a variety of substrates. That is, the reaction can employ a variety of C-nitroso dienophiles in combination with an array of dienes. With regard to the C-nitroso dienophile, a number of generic, as well as specific, compounds have been disclosed (e.g., I, Ia, Ib, Ic, Id). Furthermore, a variety of diene substrates are disclosed, including both specific and generic compounds, such as II, IIa, IIb, IIc, and IId. One skilled in the art would be aware that various combinations of these substrates could be utilized in the catalytic asymmetric C-nitroso Diels-Alder reaction disclosed herein. For example, this Diels-Alder reaction could be performed with a C-nitroso dienophile substrate, within the genus described by I or Ia, and a diene substrate, within the genus of II or IIe.

Again, one skilled in the art would be aware that there are numerous cyclic dienes that can be employed in this Diels-Alder reaction. Such compounds include those falling within the genus of diene II or within the genus of diene IIe. In one preferred embodiment, the Diels-Alder reaction is performed when the diene is an unsubstituted or substituted group selected from the following formulae:

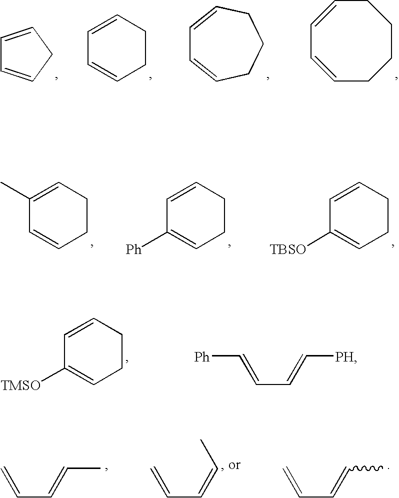

Additionally, one skilled in the art would realize this catalytic asymmetric C-nitroso Diels-Alder reaction, disclosed herein, can be performed with a variety of substrates. That is, the reaction can employ a variety of C-nitroso dienophiles in combination with an array of dienes. With regard to the C-nitroso dienophile, a number of generic, as well as specific compounds, have been disclosed (e.g., I, Ia, Ib, Ic, Id). Furthermore, a variety of diene substrates are disclosed, including both specific and generic compounds, such as II, IIa, IIb, IIc, and IId. One skilled in the art would be aware that various combinations of these substrates could be utilized in the catalytic asymmetric C-nitroso Diels-Alder reaction disclosed herein. That is, this Diels-Alder reaction can be performed with a C-nitroso dienophile substrate within the genus described by I or Ia, and a diene substrate selected from one of the following formulae:

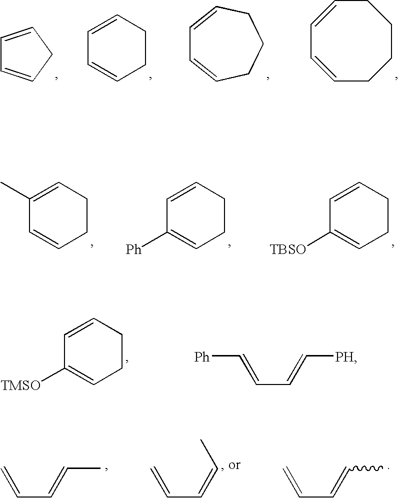

For example, the reaction could be performed between a cyclic aromatic nitroso dienophile and cyclohexa-1,3-diene or between C-nitroso dienophile I and cyclopenta-1,3-diene. In another example, the Diels-Alder reaction could be performed between C-nitroso dienophile Ia and 1-(2,5-dimethylcyclohexa-1,5-dienyl)benzene, for example. To further illustrate this point, C-nitroso dienophile Ib and the diene, (1E,3E)-1,4-diphenylbuta-1,3-diene, could be reacted. This list of possible C-nitroso dienophile and diene combinations is not exhaustive, but instead only serves to illustrate the manner in which varies dienes and dienophiles may be paired for use in the reaction disclosed herein.

Metal

This C-nitroso Diels-Alder reaction employs a chiral catalyst, which is composed of an asymmetric bidentate ligand and a metal. In one embodiment the metal is a Lewis acid. In another embodiment the metal is a transition metal Lewis acid. Examples of possible metals catalysts include, but are not limited to, copper (I), silver (V), and palladium (II). In a preferred embodiment the Lewis acid is selected from the group consisting of Cu(OTf)₂, Cu(SbF₆)₂, [CuOTf] Benzene, CuSbF₆, Cu(ClO₄), Cu(NTf₂), AgSbF₆, Pd(BF₄)₂ and CuPF₆(MeCN)₄; preferably CuPF₆(MeCN)₄ is used.

Asymmetric Bidentate Ligand

This C-nitroso Diels-Alder reaction, utilizes a chiral catalyst, which is composed of an asymmetric bidentate ligand and a metal. A variety of asymmetric bidentate ligands can be employed. For example, the Diels-Alder reaction can be performed when the ligand is a compound of formula (III): 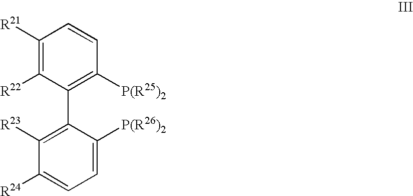
where, R²¹, R²², R²³, and R²⁴ are each independently selected from the group consisting of hydrogen, alkyl, cycloalkyl, alkoxy, halogen, heterocyclyl, aryl, arylalkyl, heteroaryl, and O-silyl. The R²¹, R²², R²³, and R²⁴ substituents are each independently selected from the group consisting of alkyl, cycloalkyl, heterocyclyl, aryl, arylalkyl, and heteroaryl. The asymmetric bidentate ligand of formula (III) can be constructed such that R²¹ and R²², together with the atoms to which they are attached, as well as R²³ and R²⁴, together with the atoms to which they are attached, form rings selected from the group consisting of cycloalkyl, heterocyclyl, aryl, and heteroaryl.

One in the art would realize that a variety of asymmetric bidentate ligands could be employed in combination with this asymmetric C-nitroso Diels-Alder reaction. Furthermore, one in the art would appreciate that by using different asymmetric bidentate ligands, it would be possible to optimize both the yield and the enantioselectivity of this Diels-Alder reaction. Furthermore, it should be apparent that a variety of asymmetric bidentate ligands could be used in combination with an assortment of C-nitroso dienophiles and dienes. To illustrate, a asymmetric bidentate ligand of formula III could be employed in this Diels-Alder reaction in combination with any of the following C-nitroso dienophile formulae:

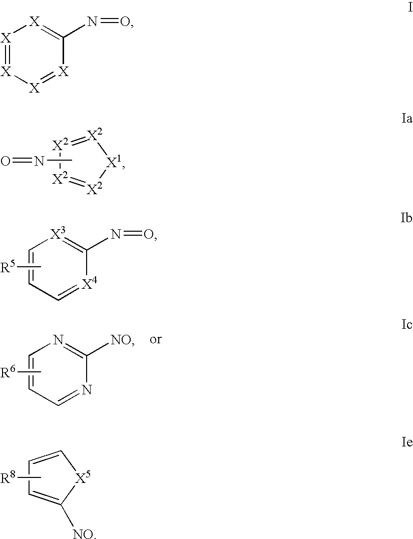

It is worth noting that this combination of asymmetric bidentate ligands and C-nitroso dienophiles is not exhaustive, but only serves to illustrate how various asymmetric bidentate ligands and various C-nitroso dienophiles might be paired in this Diels-Alder reaction. One in the art would also realize that various asymmetric bidentate ligands could be used in combination with an array of dienes.

In one preferred embodiment, the Diels-Alder reaction is performed when the asymmetric bidentate ligand is an unsubstituted or substituted compound selected from the following formulae:

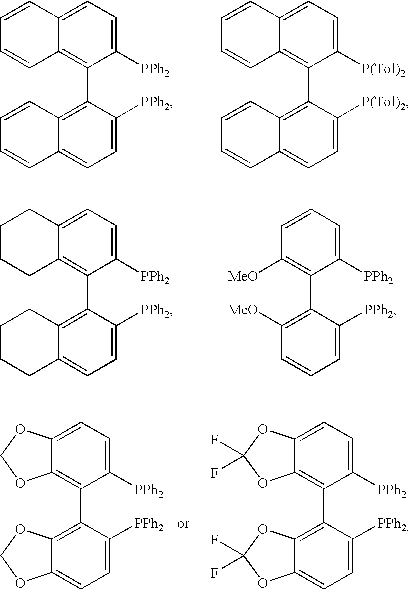

In another preferred embodiment these asymmetric bidentate ligands are employed in combination with dienophiles I, Ia, Ib, Ic, and Id.

In one embodiment, the asymmetric bidentate ligand and the metal form a complex, which serves to catalyze the asymmetric C-nitroso Diels-Alder reaction.

Reaction Conditions

The reacting step of this Diels-Alder reaction is performed in a solvent. In fact, the reaction can be performed in a variety of solvents, including, but not limited to, methylene chloride, tetrahydrofuran, and acetonitrile. Since the choice of solvent can affect the enantioselectivity, one skilled in the art would know to vary the solvent to optimize the enantioselectivity and the yield.

The Diels-Alder reaction can be performed at a variety of temperatures. However, one skilled in the art would know that changing the temperature could be used to optimize the enantioselectivity and the yield. In one embodiment, the reacting step of the Diels-Alder reaction is performed at about −85° C. to about 20° C. More preferably the reaction is carried out at about −78° C. to about 0° C.

This Diels-Alder reaction is typically performed under an inert gas. In a preferred embodiment, the reaction is performed under nitrogen or argon.

The reaction can be performed where the ratio of the dienophile (I) to the diene (II or IIe) is varied. One skilled in the art would be aware that these ratios can be varied to optimize the enantioselectivity and the yield of this Diels-Alder reaction. In one embodiment, the Diels-Alder reaction is performed where about 1.0 equivalent of the nitroso dienophile (I) and about 1.0 to about 1.5 equivalents of the diene (II or IIe) are used. More preferably, about 1.1 to about 1.2 equivalents of the diene can be used.

One skilled in the art would also realize that varying the ratio of asymmetric bidentate ligand to metal, might be necessary, in order to optimize the yield and enantioselectivity of this reaction. In a preferred embodiment, the ratio of asymmetric bidentate ligand to metal is about one to about one. Furthermore, optimizing the yield and enantioselectivity could also involve changing the number of equivalents of the chiral catalyst which are used in the reaction. In one embodiment, the Diels-Alder reaction is performed where the quantity of the asymmetric bidentate ligand and metal complex is about 0.05 to about 0.25 equivalents, more preferably, about 0.1 to about 0.15 equivalents.

### The Dihydro-1,2-oxazine Cycloadduct

The Diels-Alder reaction ultimately provides a dihydro-1,2-oxazine cycloadduct IV, in which two asymmetric centers have been formed. For example, when diene II is reacted with dienophile I, the resulting dihydro-1,2-oxazine cycloadduct is compound IV.

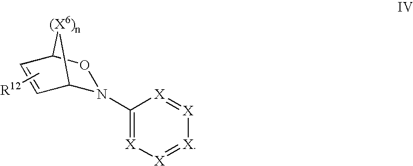

Furthermore, when dienophile Ia is reacted with diene II, the resulting dihydro-1,2-oxazine cycloadduct is compound IVa.

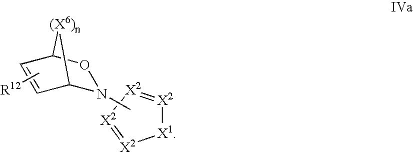

### Cleaving the Nitrogen-Oxygen Bond of the Dihydro-1,2-oxazine Cycloadduct

To generate an amino alcohol, the bond between the nitrogen and the oxygen of the dihydro-1,2-oxazine cycloadduct can be cleaved. For example, cleavage of the nitrogen-oxygen bond of compound IV provides free amino alcohol V.

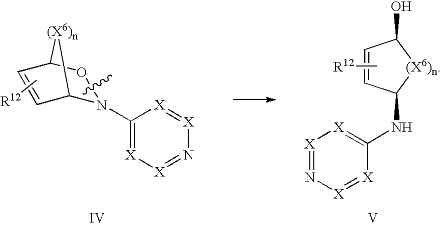

In one embodiment, the nitrogen-oxygen bond of IV is cleaved using Mo(CO)₆, NaBH₄, and aqueous MeCN.

Cleaving the Bond Between the Nitrogen of the Nitroso Group and the Carbon of the Aromatic Ring

To provide free amino alcohols, such as free amino alcohol VI, the bond between the nitrogen of the nitroso group and the carbon of the aromatic ring of compound Va is cleaved. The following scheme illustrates this process.

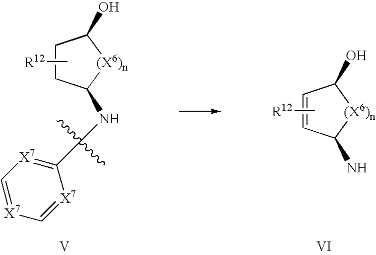

In a preferred embodiment, a process of enantioselective chemical synthesis is carried out where C-nitroso dienophile If is reacted with a 1,3-diene in the presence of an asymmetric bidentate ligand and a metal.

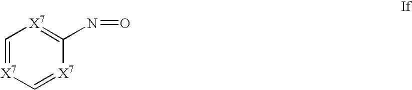

Next, the nitrogen-oxygen bond of the resulting dihydro-1,2-oxazine cycloadduct is cleaved to provide an amino alcohol precursor (such as compound Va). Next, the bond between the aromatic substituent, located on what was originally the nitro nitrogen of If, is removed from the amino alcohol precursor to provide a free amino alcohol (such as compound VI).

In another preferred embodiment, the bond between the nitrogen of the nitroso group and the carbon of the aromatic ring is cleaved by: silylating the alcohol of the amino alcohol precursor; tosylating the nitrogen which originated from the dienophile's nitroso group; methylating the nitrogen of the aromatic ring; and cleaving the bond between the aromatic ring and the nitrogen, which originated from the dienophile's nitroso group, by addition of a hydroxide base.

Another preferred embodiment involves a method of synthesizing enantiomerically enriched amino alcohols, comprising the steps of: reacting a C-nitroso dienophile and a 1,3-diene, in the presence of an asymmetric bidentate ligand and a metal, to provide a dihydro-1,2-oxazine cycloadduct; cleaving the nitrogen-oxygen bond of the dihydro-1,2-oxazine cycloadduct to provide an amino alcohol precursor; and removing the aromatic substituent from the amino alcohol precursor, located on what was originally the nitro nitrogen, to yield a free amino alcohol.

In a preferred embodiment, enantiomerically enriched amino alcohols, are synthesized by reacting If and II in the presence of an asymmetric bidentate ligand and a metal to provide IV; cleaving the nitrogen-oxygen bond of IVz to provide V; cleaving the nitrogen-aromatic ring bond of V to produce VI;

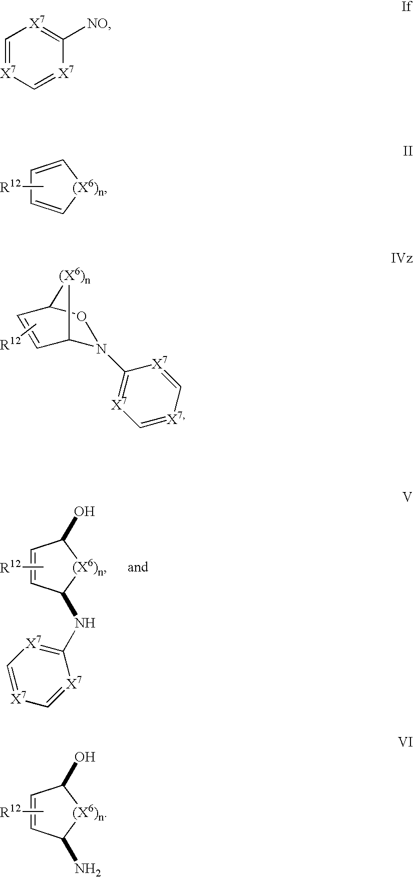

### EXAMPLES

The following examples are offered to illustrate, but not to limit, the claimed invention.

General Procedures

Unless otherwise noted, all non-aqueous reactions were carried out in oven- or flame-dried glassware under an atmosphere of dry nitrogen or argon. Except as otherwise indicated, all reactions were magnetically stirred and monitored by analytical thin-layer chromatography using Merck pre-coated silica gel plates with F₂₅₄ indicator. Visualization was accomplished by UV light (256 nm), potassium permanganate, phosphomolybdic acid, and/or ferric chloride solution. Flash column chromatography was performed using silica gel 60 (mesh 230-400) supplied by E. Merck. Yields refer to chromatographically and spectrographically pure compounds, unless otherwise noted.

Commercial grade reagents and solvents were used without further purification except as indicated below. Diethyl ether (Et₂O), tetrahydrofuran (THF), and toluene (PhCH₃) were distilled from sodium-benzophenone ketyl under an atmosphere of dry argon. Dichloromethane (CH₂Cl₂) and triethylamine (Et₃N) were distilled from calcium hydride, under an atmosphere of dry nitrogen. Brine refers to a saturated aqueous solution of NaCl. All other reagents and starting materials, unless otherwise noted, were purchased from commercial vendors and used without further purification. 2-Cyclohepetene-1-one was distilled under P₂O₅. 1,4-Dioxaspiro[4,5]dec-6-en-8-one was prepared according to Kerr et al. See Kerr, W. J.; McLaughlin, M.; Morrison, A. J.; Pauson, P. L. Org. Lett, 2001, 3, 2945-2948.

Infrared spectra were recorded as thin films on sodium chloride plates using a Nicolet 20 SXB FTIR. ¹H NMR and ¹³C NMR spectra were recorded on a Bruker Avance 400 (400 MHz ¹H, 100 MHz ¹³C), a Bruker Avance 500 (500 MHz ¹H, 125 MHz ¹³C). Chemical shift values (δ) are reported in ppm relative to residual chloroform (δ 7.27 ppm for ¹H; δ 77.23 ppm for ¹³C), methanol (δ 3.30 ppm for ¹H; δ 49.0 ppm for ¹³C), Me₄Si (δ 0.0 ppm) or DMSO (δ 2.50 ppm for ¹H; δ 39.5 ppm for ¹³C). The proton spectra are reported as follows δ (multiplicity, number of protons, coupling constant J). Multiplicities are indicated by s (singlet), d (doublet), t (triplet), q (quartet), p (pentet), h (heptet), m (multiplet) and br (broad).

### Example 1

### General Procedure for the catalytic asymmetric C-nitroso Diels-Alder reaction (Reaction between 6-methyl-2-nitrosopyridine and 1,3-cyclopentyl Diene)

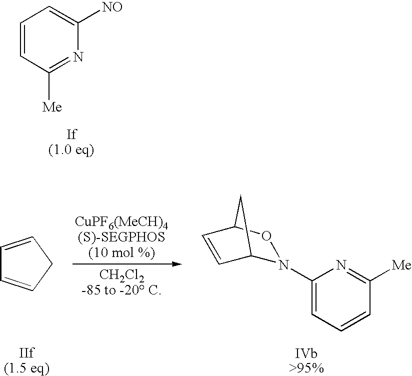

To a Schlenk tube was added Copper (I) (CH₃CN)₄ PF₆ (18.6 mg, 0.05 mmol) and (S)-(−) SEGPHOS (32.1 mg, 0.0505 mmol). The mixture was dried under vacuum for 10 min and then anhydrous CH₂Cl₂ (4 mL) was added. Next, the mixture was stirred for 1 h. The clear solution was then cooled to −85° C. and If, dissolved in anhydrous CH₂Cl₂ (1 mL), was added dropwise. After the resulting dark blue solution was stirred for 10 min, diene IIf, dissolved in anhydrous CH₂Cl₂, was added dropwise over a 1 h period. The reaction mixture was gradually warmed to −20° C. over a 5 h period and was then stirred at −20° C. for an additional hour. The crude product was purified by silica gel chromatography to afford C-nitroso Diels-Alder adduct IVb. Dihydro-1,2-oxazine cycloadduct IVb was purified by flash column chromatography with elution by (4:1 hexane:ethylacetate) to provide a yellowish crystal in >95% yield and 90% ee. TLC R_{f} 0.7 (EtOAc/Hexanes, 1:3); [o]_{D}²⁸−309.0° (c=1.18, CHCl₃); R_{f}0.7 (EtOAc/Hexanes, 1:3); FTIR (CD₃Cl) υₘₐₓ 3012, 2958, 1588, 1578, 1452, 1330, 1307, 1231, 926, 856, 789 cm⁻¹; ¹H NMR (500 MHz, CD₃Cl) δ 7.38 (t, J=8.0 Hz, 1H), 6.56-6.65 (m, 2H), 6.30-6.31 (m, 1H), 6.01-6.11 (m, 1H), 5.50 (br s, 1H), 5.19 (br s, 1H), 2.43 (s, 3H), 2.15 (d, J=8.5 Hz, 1H), 1.78 (d, J=8.5 Hz, 1H); ¹³C NMR (125 MHz, CD₃Cl) δ 163.2, 156.4, 137.6, 135.0, 132.4, 116.5, 109.0, 82.8, 66.8, 47.9, 24.3; MS (Cl) Exact Mass Calculated for C₁₁H₁₃N₂O (M+H)⁺: 189.1. Found: 189.1. Enantiometric excess was determined by HPLC with Chiralcel OD-H column (95:5 hexane:2-propanol), 1.0 mL/min; major enantiomer tᵣ=7.6 min, minor enantiomer tᵣ=9.7 min.

Additional results for the reaction between 6-methyl-2-nitrospyridine and various other cyclic 1,3-dienes are provided in Table 1 below.

TABLE 1
Reaction of 6-methyl-2-nitrosopyridine with various cyclic 1,3-dienes.

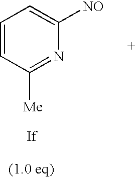

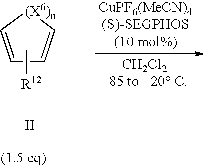

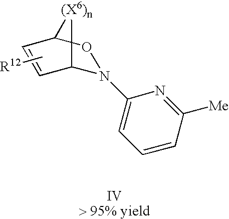

| | | pro- | | | | pro- | |
|---|---|---|---|---|---|---|---|
| entry | diene | duct | ee(%)^{a} | entry | diene | duct | ee(%)^{a} |
| 1 | 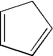 | IVb | 90 | 5 | 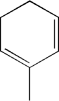 | IVf | 94 |
| 2 | 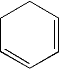 | IVc | 92 | 6 | 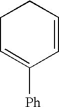 | IVg | 97 |
| 3 | 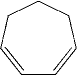 | Ivd | 72 | 7 | 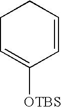 | IVh | 92 |
| 4 | 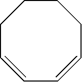 | IVe | 4 | 8^{b} | 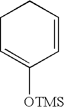 | IVi | 77 |

^{a}Determined by chiral HPLC.
^{BINAP was used.}

Table 1 highlights the ability of the Diels-Alder reaction, disclosed herein, to function with a variety of 1,3-dienes. The enantiomeric excesses and the yields shown correspond to the reaction of 6-methyl-2-nitrosopyridine with 8 different 1,3-dienes. In each case, the yields were above 95% and enantioselectivity was achieved. For instance, in entry 6, 1-(cyclohexa-1,5-dienyl)benzene provided an enantiomeric excess (ee) of 97%, while use of 1,3-cyclohexadiene afforded an ee of 92%. Even (1Z, 3Z)-cycloocta-1,3-diene provided an enantiomeric excess. Thus Table, 1 provides an example of how this cycloaddition can be successfully applied to a wide variety of dienes. However, it is important to note that the data in this table is illustrative only and is in no way exhaustive. Spectroscopic data for some of the dihydro-1,2-oxazine cycloadducts, contained within Table 1, has been provided in the following text as Examples 2 to 6.

### Example 2

### Reaction of 6-methyl-2-nitrosopyridine and 1,3-cyclohexyl diene

This reaction was carried out using the general procedure, described in Example 1. Dihydro-1,2-oxazine cycloadduct IVd was purified by flash column chromatography with elution by (4:1 hexane:ethylacetate) to a white crystal. TLC R_{f} 0.7 (EtOAc/Hexanes, 1:3); [α]_{D}²⁸−209.0° (c=1.06, CHCl₃); R_{f} 0.7 (EtOAc/Hexanes, 1:3); FTIR (CD₃Cl) υₘₐₓ 2965, 2935, 1588, 1577, 1448, 1265, 912 cm⁻¹; ¹H NMR (400 MHz, CD₃Cl) δ 7.39 (t, J=8.0 Hz, 1H), 6.71 (d, J=8.2 Hz, 1H), 6.63 (d, J=7.4 Hz, 1H), 6.46-6.50 (m, 1H), 6.26-6.30 (m, 1H), 5.30-5.32 (m, 1H), 4.68-4.72 (m, 1H), 2.42 (s, 3H), 2.20-2.30 (m, 2H), 1.56-1.62 (m, 1H), 1.35-1.41 (m, 1H); ¹³C NMR (100 MHz, CD₃Cl) δ 163.9, 156.3, 137.6, 131.8, 130.8, 116.1, 108.1, 69.7, 52.5, 24.4, 24.3, 20.6; MS (Cl) Exact Mass Calculated for C₁₂H₁₅N₂O (M+H)⁺: 203.1. Found: 203.1. Enantiometric excess was determined by HPLC with Chiralcel OD-H column (95:5 hexane:2-propanol), 1.0 mL/min; major enantiomer tᵣ=8.4 min, minor enantiomer tᵣ=7.7 min.

### Example 3

### Reaction of 6-methyl-2-nitrosopyridine with (1Z,3Z)-cyclohepta-1,3-diene

This reaction was carried out using the general procedure, described in Example 1. Dihydro-1,2-oxazine cycloadduct IVd was purified by flash column chromatography with elution by (9:1 hexane:ethylacetate) to provide a white crystal. TLC R_{f} 0.7 (EtOAc/Hexanes, 1:4); [α]_{D}²⁸−134.7° (c=1.16, CHCl₃); FTIR (CD₃Cl) υₘₐₓ 2937, 1577, 1449, 1285, 1230, 1155, 975, 890, 793 cm⁻¹; ¹H NMR (400 MHz, CD₃Cl) δ 7.42 (t, J=8.1 Hz, 1H), 6.80 (d, J=8.2 Hz, 1H), 6.60 (d, J=7.4 Hz, 1H), 6.15-6.24 (m, 1H), 6.02-6.06 (m, 1H), 5.30-5.38 (m, 1H), 4.79-4.80 (m, 1H), 2.41 (s, 3H), 1.91-2.18 (m, 3H), 1.72-1.75 (m, 1H), 1.58-1.62 (m, 1H), 1.38-1.48 (m, 1H); ¹³C NMR (100 MHz, CD₃Cl) δ 163.6, 156.4, 137.8, 130.5, 125.7, 115.6, 107.7, 73.5, 57.1, 31.8, 27.3, 24.4, 18.8, 12.7. Enantiometric excess was determined by HPLC with Chiralcel OD-H column (95:5 hexane:2-propanol), 1.0 mL/min; major enantiomer tᵣ=6.9 min, minor enantiomer tᵣ=6.2 min.

### Example 4

### Reaction of 6-methyl-2-nitrosopyridine with 2-methylcyclohexa-1,3-diene

This reaction was carried out using the general procedure, described in Example 1. Dihydro-1,2-oxazine cycloadduct IVf was purified by flash column chromatography with elution by (5:1 hexane:ethylacetate) to provide a colorless oil. TLC R_{f} 0.7 (EtOAc/Hexanes, 1:3); [α]_{D}²⁸−150.9° (c=1.10, CHCl₃); R_{f} 0.7 (EtOAc/Hexanes, 1:4); FTIR (CD₃Cl) υₘₐₓ 2964, 2934, 1588, 1576, 1450, 1264, 1230, 914, 885, 789 cm⁻¹; ¹H NMR (400 MHz, CD₃Cl) δ 7.39 (t, J=8.0 Hz, 1H), 6.72 (d, J=8.2 Hz, 1H), 6.62 (d, J=7.4 Hz, 1H), 6.02-6.04 (m, 1H), 5.11-5.12 (m, 1H), 4.67-4.69 (m, 1H), 2.42 (s, 3H), 2.18-2.23 (m, 2H), 1.68 (s, 3H), 1.57-1.63 (m, 1H), 1.33-1.36 (m, 1H); ¹³C NMR (100 MHz, CD₃Cl) δ 164.2, 156.0, 141.5, 137.6, 108.2, 70.7, 56.7, 25.4, 24.3, 20.6, 20.2. MS (Cl) Exact Mass Calculated for C₁₃H₁₇N₂O (M+H)⁺: 217.1. Found: 217.1. Enantiometric excess was determined by HPLC with Chiralcel OD-H column (99:1 hexane:2-propanol), 1.0 mL/min; major enantiomer tᵣ=15.3 min, minor enantiomer t, =11.1 min.

### Example 5

### Reaction of 6-methyl-2-nitrosopyridine with 1 (cyclohexa-1,5-dienyl)benzene

This reaction was carried out using the general procedure, described in Example 1. Dihydro-1,2-oxazine cycloadduct IVg was purified by flash column chromatography with elution by (9:1 hexane:ethylacetate) to provide a colorless oil. TLC R_{f} 0.7 (EtOAc/Hexanes, 1:5); [α]_{D}²⁸+113.0° (c=1.10, CHCl₃); FTIR (CD₃Cl) υₘₐₓ 3056, 2966, 2934, 158, 1575, 1449, 1339, 1312, 1267, 1226, 1156, 961, 928, 886, 789 cm⁻¹; ¹H NMR (400 MHz, CD₃Cl) δ 7.56 (d, J=8.0 Hz, 2H), 7.22-7.33 (m, 4H), 6.73 (d, J=8.2 Hz, 1H), 6.64-6.66 (m, 1H), 6.54 (d, J=7.4 Hz, 1H), 5.78-5.80 (m, 1H), 4.88-4.90 (m, 1H), 2.43 (s, 3H), 2.29-2.43 (m, 2H), 1.65-1.71 (m, 1H), 1.41-1.48 (m, 1H); ¹³C NMR (100 MHz, CD₃Cl) δ 163.4, 155.9, 142.8, 137.7, 136.2, 128.3, 127.9, 125.6, 122.5, 116.2, 107.9, 70.1, 54.4, 24.7, 24.1, 21.0. MS (Cl) Exact Mass Calculated for C₁₈H₁₉N₂O (M+H)⁺: 279.1. Found: 279.1. Enantiometric excess was determined by HPLC with Chiralcel OD-H column (98:2 hexane:2-propanol), 1.0 mL/min; major enantiomer tᵣ=14.9 min, minor enantiomer tᵣ=10.0 min.

### Example 6

### Reaction of 6-methyl-2-nitrosopyridine with tert-butyl(cyclohexa-1,5-dienyloxy)dimethylsilane

This reaction was carried out using the general procedure, described in Example 1. Dihydro-1,2-oxazine cycloadduct IVh was purified by flash column chromatography with elution by (9:1:0.02 hexane:ethylacetate:triethylamine) to provide a white crystal. TLC R_{f} 0.7 (EtOAc/Hexanes/triethyamine, 1:5:0.02); [α]_{D}²⁶−74.4° (c=1.12, CHCl₃); FTIR (CD₃Cl) υₘₐₓ 3067, 2927, 2854, 1635, 1756, 1448, 1355, 1210, 905 cm⁻¹; ¹H NMR (400 MHz, CD₃Cl) δ 7.65 (t, J=7.3 Hz, 1H), 7.02 (d, J=8.1 Hz, 1H), 6.89 (d, J=7.3 Hz, 1H), 5.40-5.43 (m, 1H), 5.31-5.35 (m, 1H), 5.05-5.09 (m, 1H), 2.66 (s, 3H), 2.44-2.49 (m, 2H), 2.01-2.06 (m, 1H), 1.63-1.68 (m, 1H), 1.05 (s, 9H), 0.29 (s, 3H), 0.26 (s, 3H); ¹³C NMR (100 MHz, CD₃Cl) δ 164.0, 156.2, 153.4, 137.5, 116.3, 108.1, 100.3, 72.0, 58.5, 26.3, 25.3, 24.3, 21.1, 17.7, −4.57, −5.75. MS (Cl) Exact Mass Calculated for C₁₈H₂₉N₂O₂Si (M+H)⁺: 333.2. Found: 333.2. Enantiometric excess was determined by HPLC with Chiralcel OD-H column (95:5 hexane:2-propanol), 1.0 mL/min; major enantiomer tᵣ=8.0 min, minor enantiomer tᵣ=6.0 min.

TABLE 2
Reaction between 1,3-cyclohexyl diene and various nitroso-
Zpyridine compounds.

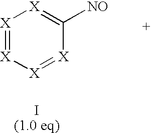

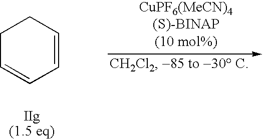

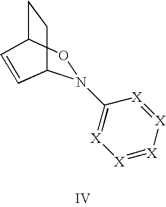

| entry | Nitroso compounds | Product | yield (%)^{a} | ee (%)^{b} |
|---|---|---|---|---|
| 1 | 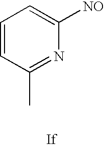 | IVc | 98 | 87 |
| 2 | 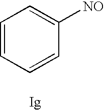 | lVj | 95 | 59 |
| 3 | 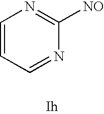 | IVk | 96 | 34 |
| 4 | 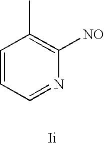 | IVl | 98 | 53 |
| 5 | 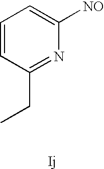 | IVm | 98 | 86 |
| 6 | 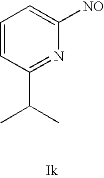 | IVn | 98 | 77 |
| 7 | 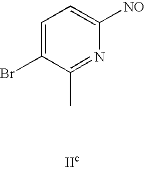 | IVo | >95 | 94 |
| 8 | 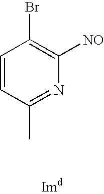 | IVp | >95 | 80 |
| 9 | 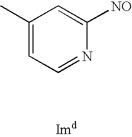 | IVq | 95 | 63 |

^{a}Isolated yield
^{b}Determined by chiral HPLC
^{c}SEGPHOS was employed as the chiral ligand; this reaction was run at −78 to −30° C.
^{d}This reaction was run at −78 to −30° C.

Table 2 demonstrates that this Diels-Alder reaction can function with an assortment of substituted and unsubstitued C-nitroso dienophiles. In each case, the catalytic asymmetric C-nitroso Diels-Alder reaction provided enantioselectivity, with the enantiomeric excess ranging from 34 to 87%. Furthermore, each reaction afforded the dihydro-1,2-oxazine cycloadduct IV in high yield. It is again important to note that this table is illustrative and is in no way exhaustive. Spectroscopic data for some of the dihydro-1,2-oxazine cycloadducts, contained within Table 2, has been provided in the following text as Examples 7 to 9.

### Example 7

### Reaction between 1,3-cyclohexadiene and 2-nitrosopyridine

This reaction was carried out using the general procedure, described in Example 1, to provide compound IVj. ¹H NMR (500 MHz, CD₃Cl) δ 8.21 (d, J=1.8 Hz, 1H), 7.51 (dd, J=7.2 Hz, J=7.2 Hz 1H), 6.92 (d, J=6.7 Hz, 1H), 6.77 (dd, J=0.7 Hz, J=0.7 Hz 1H), 6.46-6.48 (m, 1H), 6.32-6.33 (m, 1H), 5.27-5.30 (m, 1H), 4.72-4.75 (m, 1H), 2.22-2.28 (m, 2H), 1.57-1.62 (m, 1H), 1.38-1.44 (m, 1H). Enantiometric excess was determined by HPLC with Chiralcel OD-H column (95:5 hexane:2-propanol), 1.0 mL/min; major enantiomer tᵣ=12.9 min, minor enantiomer tᵣ=9.7 min

### Example 8

### Reaction Between 1,3-cyclohexadiene and 3-methyl-2-nitorosopyridine

This reaction was carried out using the general procedure, described in Example 1, to provide compound IVI. ¹H NMR (500 MHz, CD₃Cl) δ 8.08 (d, J=0.9 Hz, 1H), 7.34 (d, J=7.3, 1H), 6.83 (dd, J=7.4 Hz, J=4.8 Hz, 1H), 6.54-6.59 (m, 1H), 6.49-6.54 (m, 1H), 4.74-4.79 (m, 1H), 4.57-4.62 (m, 1H), 2.35 (s, 3H), 2.23-2.29 (m, 2H), 1.54-1.62 (m, 1H), 1.40-1.45 (m, 1H); ¹³C NMR (100 MHz, CD₃Cl) δ 161.0, 144.0, 139.3, 133.8, 131.0, 126.3, 118.9, 69.5, 51.1, 24.7, 21.2, 19.0. Enantiometric excess was determined by HPLC with Chiralcel OD-H column (95:5 hexane:2-propanol), 1.0 mL/min; major enantiomer tᵣ=7.4 min, minor enantiomer tᵣ=6.4 min.

### Example 9

### Reaction between 1,3-cyclohexadiene and 2-isopropyl-6-nitrosopyridine

This reaction was carried out using the general procedure, described in Example 1 to provide compound IVn. ¹H NMR (500 MHz, CD₃Cl) δ 7.43 (t, J=7.5 Hz, 1H), 6.73 (d, J=8.0, 1H), 6.66 (d, J=7.5 Hz, 1H), 6.46 (dd, J=6.5 Hz, J=6.5 Hz, 1H), 6.29 (dd, J=7.0 Hz, J=7.0 Hz, 1H), (m, 1H), 5.36-5.40 (m, 1H), 4.67-4.71 (m, 1H), 2.86-2.95 (m, 1H), 2.20-2.31 (m, 2H), 1.55-1.73 (m, 1H), 1.37-1.43 (m, 1H), 1.26 (d, J=7.0 Hz, 6H); ¹³C NMR (125 MHz, CD₃Cl) δ 165.0, 163.6, 137.6, 132.2, 130.7, 113.4, 108.5, 69.6, 52.0, 35.9, 24.4, 22.7, 22.2, 20.5. Enantiometric excess was determined by HPLC with Chiralcel AD-H column (95:5 hexane:2-propanol), 1.0 mL/min; major enantiomer tᵣ=13.2 min, minor enantiomer tᵣ=14.4 min.

TABLE 3
Reaction of 2-nitrosopyridine with a variety of metal sources,
solvents and temperatures.

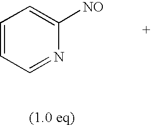

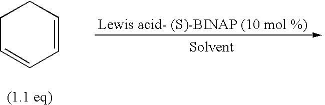

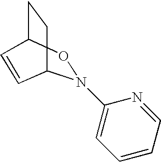

| | | | | yield | ee | |
|---|---|---|---|---|---|---|
| entry | Lewis acid | temp. (° C.) | solvent | (%)^{a} | (%)^{b} | config^{c} |
| 1 | Cu(OTf)₂ | RT | CH₂Cl₂ | 66 | 7 | A |
| 2 | Cu(OTf)₂ | −20 | CH₂Cl₂ | 76 | 4 | A |
| 3 | Cu(OTf)₂ | −78 | CH₂Cl₂ | 91 | 13 | B |
| 4 | Cu(OTf)₂ | −90 | CH₂Cl₂ | 91 | 22 | B |
| 5^{d} | Cu(SbF₆)₂ | −78 | CH₂Cl₂ | 73 | 32 | A |
| 6^{d} | Cu(SbF₆)₂ | −90 | CH₂Cl₂ | 84 | 7 | A |
| 7^{d,e} | Cu(SbF₆)₂ | −78 | CH₂Cl₂ | 84 | 6 | A |
| 8 | CuPF₆(MeCN)₄ | −78 | CH₂Cl₂ | 96 | 40 | B |
| 9 | CuPF₆(MeCN)₄ | −85 to −30 | CH₂Cl₂ | 95 | 59 | B |
| 10 | CuPF₆(MeCN)₄ | −85 to −30 | THF | 95 | 59 | B |
| 11 | CuPF₆(MeCN)₄ | −85 to −30 | MeCN | 94 | 0 | |
| 12 | [CuOTf] Benzene | −85 to −30 | MeCN | 94 | 43 | B |
| 13 | CuSbF₆ | −85 to −30 | CH₂Cl₂ | 96 | 58 | B |
| 14 | AgSbF₆ | −85 to −30 | CH₂Cl₂ | 92 | 18 | A |
| 15^{f} | Pd(BF₄) | −85 to −30 | CH₂Cl₂ | 94 | 6 | B |
| 16^{g} | CuPF₆(MeCN)₄ | −85 to −30 | CH₂Cl₂ | 96 | 59 | B |
| 17^{h} | CuPF₆(MeCN)₄ | −85 to −30 | CH₂Cl₂ | 96 | 62 | A |
| 18ⁱ | CuPF₆(MeCN)₄ | −78 to −30 | CH₂Cl₂ | 95 | 67 | B |
| 19 | CuPF₆(MeCN)₄ | −78 to −30 | CH₂Cl₂ | 95 | 68 | B |

^{a}Isolated yield
^{b}Determined by chiral HPLC
^{c}HPLC retention time (HPLC conditions cited on experimental), config A: retention time (9.7 min), config B: retention time (13.9 min)
^{d}catalysis and substrate was aged at RT.
^{e}Prepared by CuCl₂ and AgSbF₆.
^{f}Prepared by PdCl₂(MeCN)₄ and AgSbF₆.
^{g}used 2 eq of diene.
^{h}used (R)-Tol-BINAP.
ⁱused 20 mol% BINAP.
^{j}used 20 mol% BINAP.

Table 3 provides a series of results, which correspond to the reaction of 2-nitrosopyridine and 1,3-cyclohexadiene, under a variety of reaction conditions. The results provided in this table have been obtained with several different solvents, numerous Lewis acid metals, and a range of different temperatures. In each case, this Diels-Alder reaction provided good yields and in all but one example, enantioselectivity was achieved. Table 1 demonstrates that this Diels-Alder reaction can be performed under a variety of conditions and with a variety of reagents. Furthermore, one skilled in the art would realize that this reaction can be optimized for specific dienes and dienophiles by changing the types of reaction conditions which are shown in this table. That is, one skilled in the art would understand that optimization of yields and enantioselectivies can be achieved by changing these types of conditions. The data and parameters shown are only illustrative and are in no way limiting or exhaustive.

TABLE 4
Diels-Alder reaction between 6-methyl-2-nitrosopyridine and
various acyclic 1,3-dienes.

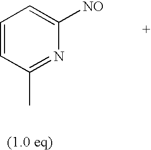

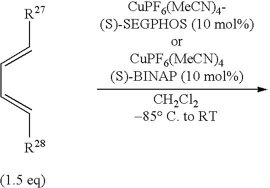

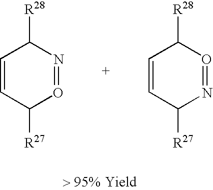

| | | | | | |
|---|---|---|---|---|---|
| | | | | | regio of cis/ |
| entry | diene | ee | entry | diene | trans selectivity (% ee) |

| | | | | | | |
|---|---|---|---|---|---|---|
| 1 | 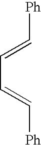 | ca. 20 SEGPHOS | 2 | 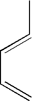 | no cat. BINAP | 1.3:1 7(14):1(>60) |
| | | | 3 | 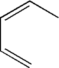 | no cat. BINAP | 1.3:1 7(14):1(>60) |
| | | | 4 | 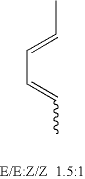 | no cat. BINAP | 4:1 1.2(0):1(38) |

Table 4, demonstrates the ability of this Diels-Alder reaction to utilize acyclic diene substrates. In all but one case, the reaction provided a cyclo-adduct product with an enantiomeric excess (no such selectivity was obtained for the cis product in entry 4). The substrates shown in this table are only illustrative and are in no way limiting or exhaustive.

R²⁷ and R²⁸ are each independently selected from the group consisting of alkyl, cycloalkyl, alkoxy, alkylamino, alkylthio, aryl, arylalkyl, heterocyclyl, heteroaryl, halogen, silyloxy, carboxylic acid, ester, alkene, azide, amine, hydroxyl, imine, ketone, thiole, amide, silyl, nitrile, sulfoxide, sulfone, sulfonamide and nitroso.

### Example 15

### General Reaction for the Cleavage of the Cycloadduct Nitrogen-Oxygen Bond

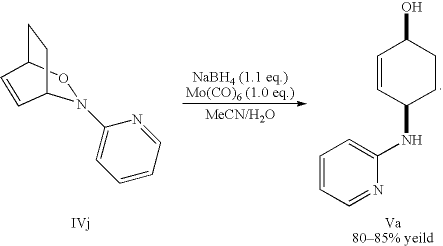

To a solution of IVj (6.06 g, 30 mmol) in MeCN (150 mL) and water (10 mL) was added NaBH₄ (1.21 g, 33 mmol) and Mo(CO)₆ (7.9 g, 30 mmol). This suspension was stirred at 50° C. for 5 h. The resulting muddy reaction mixture was filtered and the filtrate was dried over Na₂SO₄. The filtrate was concentrated under reduced pressure and the residue was purified by silica gel chromatography to provide amino alcohol Va. Amino alcohol Va was purified by flash column chromatography with elution by (9:1 hexane:ethylacetate) to provide a white crystal in 80-85% yield. TLC R_{f} 0.4 (EtOAc/Hexanes, 1:1); ¹H NMR (400 MHz, CD₃Cl) δ 8.21 (d, J=3.5 Hz, 1H), 7.37-7.43 (m, 1H), 6.56 (dd, J=6.7 Hz, J=5.1 Hz 1H), 6.38 (d, J=8.4 Hz, 1H), 5.84-5.92 (m, 2H), 4.31 (br d, J=3.6 Hz 1H), 4.21 (br s, 1H), 4.16 (br s, 1H), 1.76-1.92 (m, 4H).

### Example 16

### Cleavage of the Cycloadduct Nitrogen-Oxygen Bond

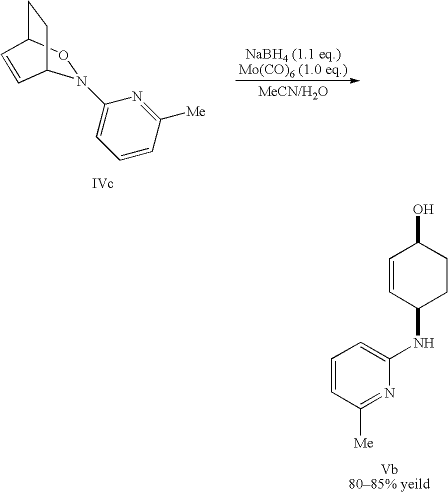

This reaction was carried out using the general procedure, described in Example 15, to provide compound Vb. Amino alcohol Vb was purified by flash column chromatography with elution by (9:1 hexane:ethylacetate) to provide a white crystal. TLC R_{f} 0.4 (EtOAc/Hexanes, 1:1); ¹H NMR (500 MHz, CD₃Cl) δ 7.33 (t, J=7.5 Hz, 1H), 6.37 (d, J=7.3 Hz, 1H), 6.13 (d, J=8.3 Hz, 1H), 4.56 (br s, 1H), 4.15 (br s, 1H), 4.12 (br s, 1H), 2.29 (s, 3H), 1.72-1.90 (m, 4H).

### Example 17

### Method for Removing the Aromatic Group from the Amino Alcohol Precursor

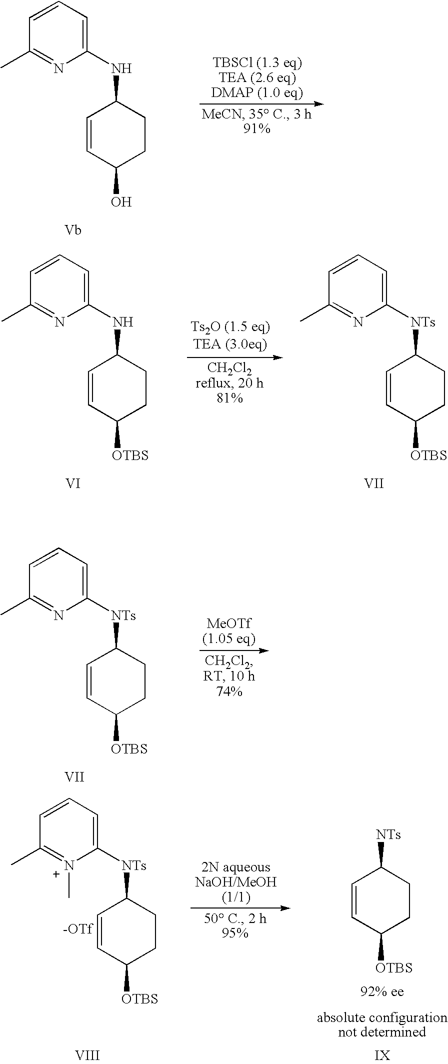

This scheme describes one method of removing the aromatic group, in this case pyridine, from the nitrogen of the amino alcohol precursor (Vb). First, the hydroxyl group of Vb is silylated with TBS, to provide VI. Compound VI is then tosylated, yielding tosyl amine VII. In the next step, the pyridine nitrogen is methylated with methyl triflate, generating compound VIII. Finally, the methylated pyridine group is removed with the addition of an aqueous base, in this case sodium hydroxide, providing the free amino alcohol IX. In general, this is a novel and simple route for the removal of the pyridine, which, as shown, can be carried out to provide a high yield of IX.

TABLE 5
Preparation of Nitrosopyridine Derivatives.

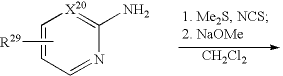

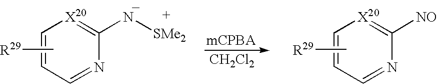

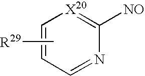

| Entry | Product | Yield % |
|---|---|---|
| 1 | 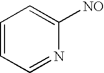 | 45 |
| 2 | 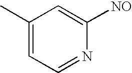 | 32 |
| 3 | 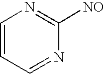 | 58 |
| 4 | 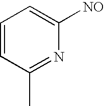 | 34 |
| 5 | 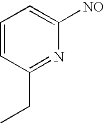 | 30 |
| 6 | 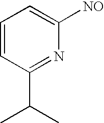 | 7 |
| 7 | 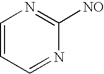 | 4 |

Table 5 illustrates a number of nitrosopyridine compounds that were synthesized from the corresponding amines using the method reported by Taylor et al. See Taylor et al., JOC 1982, 47, 552-555. See also Taylor et al., JOC 1986, 51,101-102.

R²⁹ represents 0 to 4 substituents each of which is independently selected from the group consisting of alkyl, cycloalkyl, alkoxy, alkylamino, alkylthio, aryl, arylalkyl, heterocyclyl, heteroaryl, halogen, silyloxy, carboxylic acid, ester, alkene, azide, amine, hydroxyl, imine, ketone, thiole, amide, silyl, nitrile, sulfoxide, sulfone, sulfonamide and nitroso.

X²⁰ is selected from the group consisting of —CR³⁰— and —N—. R³⁰ is selected from the group consisting of alkyl, cycloalkyl, alkoxy, alkylamino, alkylthio, aryl, arylalkyl, heterocyclyl, heteroaryl, halogen, silyloxy, carboxylic acid, ester, alkene, azide, amine, hydroxyl, imine, ketone, thiole, amide, silyl, nitrile, sulfoxide, sulfone, sulfonamide and nitroso.

Characterization data for the product of Table 5, Entry 2: Purification by flash column chromatography with elution by (4:1 hexane:ethylacetate) provided as a white crystal (99% yield, 63% ee); TLC R_{f} 0.7 (EtOAc/Hexanes, 1:3); [α]_{D}³¹−126.3 (c=0.92, CHCl₃); FTIR (CD₃Cl) υₘₐₓ 3055, 2935, 1601, 1559, 1448, 1409, 1289, 1265, 1163, 1070, 955, 908 cm⁻¹;

¹H NMR (400 MHz, CD₃Cl) δ 8.06 (d, J=5.0 Hz, 1H), 6.75 (s, 1H), 6.61 (br d, J=5.0 Hz, 1H), 6.48 (ddd, J=7.8 Hz, J=5.8 Hz, J=1.7 Hz, 1H), 6.32 (ddd, J=7.4 Hz, J=5.8 Hz, J=1.5 Hz, 1H), 5.25-5.29 (m, 1H), 4.70-4.40 (m, 1H), 2.22-2.30 (m, 5H), 1.57-1.63 (m, 1H), 1.34-1.44 (m, 1H); ¹³C NMR (100 MHz, CD₃Cl) δ 164.2, 148.7, 147.0, 131.9, 130.9, 118.0, 111.8, 70.0, 52.2, 24.3, 21.3, 20.6; Enantiometric excess was determined by HPLC with Chiralcel OD-H column (95:5 hexane:2-propanol), 1.0 mL/min; major enantiomer tᵣ=8.0 min, minor enantiomer tᵣ=9.7 min.

TABLE 6
Reaction of 2-methyl-6-nitroso-pyridine with a
variety of chiral phosphine ligands.

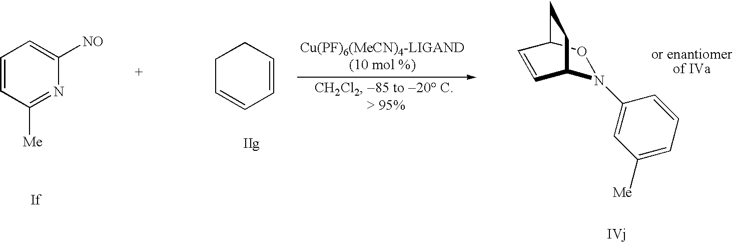

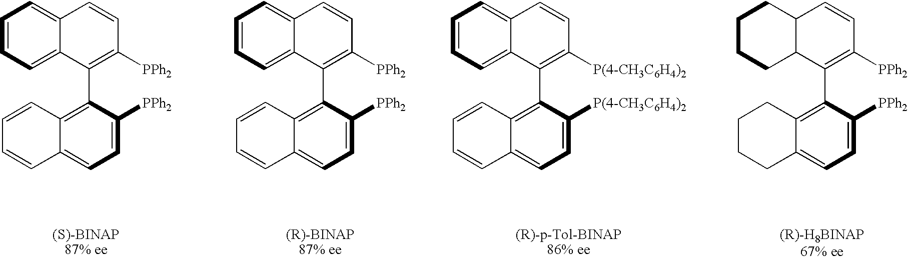

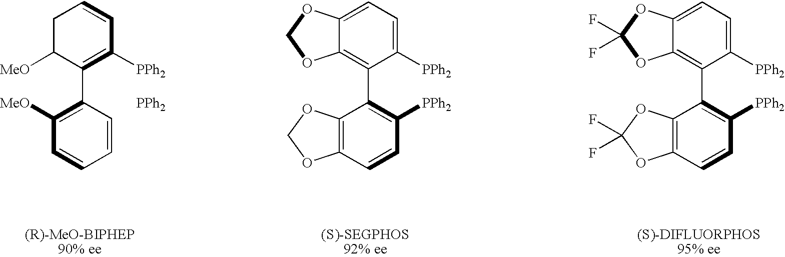

Table 6 provides a survey of various chiral phosphine ligands. Although (R)-p-Tol-BINAP showed almost no change in enantioselectivity, increased selectivity was observed using DIFLUORPHOS, which provided 95% ee.

TABLE 7
Reaction of 2-methyl-6-nitroso-pyridine with a variety of dienes
and (S)-DIFLUORPHOS.

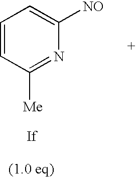

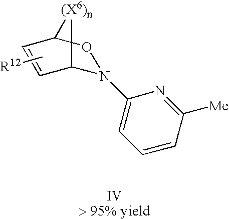

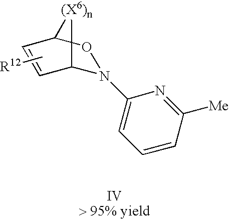

| entry | diene | product | ee(%)^{a} |
|---|---|---|---|
| 1 | 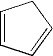 | IVb | 92 |
| 2 | 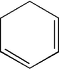 | IVc | 95 |
| 3 | 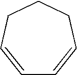 | IVd | 80 |
| 4 | 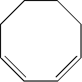 | IVe | 4 |
| 5 | 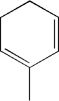 | IVf | 95 |
| 6 | 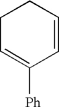 | IVg | 98 |
| 7^{a,b} | 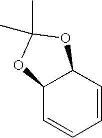 | IVj | 88 |

^{a}BINAP was used.
^{b}Temperature was warmed to room temperature.

Each of the reactions in Table 7 proceeded to completion and the desired cyclic adduct IV was the only detectable product. The regioselectivity of the reaction with the 2-substituted 1,3-cyclohexadienes provided a single regioisomer.

Characterization data for IVj: purification by flash column chromatography with elution by (4:1 hexane:ethylacetate) provided as a white crystal (99% yield, 88% ee); TLC R_{f} 0.7 (EtOAc/Hexanes, 1:3); [α]_{D}³¹−126.3 (c=0.92, CHCl₃); FTIR (CD₃Cl) υₘₐₓ 2953, 2859, 1639, 1577, 1450, 1363, 1252, 1222 cm⁻¹; ¹H NMR (400 MHz, CD₃Cl) δ 7.61 (t, J=7.8 Hz, 1H), 7.03 (d, J=8.2 Hz, 1H), 6.85 (d, J=7.4 Hz, 1H), 5.35 (dd, J=6.6 Hz, J=2.6 Hz, 1H), 5.15-5.21 (m, 1H), 4.43 (d, J=6.6 Hz, 1H), 2.61 (s, 3H), 2.15 (dd, J=12.9 Hz, J=3.3 Hz, 1H), 1.76 (dd, J=3.0 Hz, J=13.0 Hz, 1H), 1.51 (s, 3 H), 1.12 (s, 3H), 1.00 (s, 9H), 0.26 (s, 3H), 0.24 (s, 3H); ¹³C NMR (100 MHz, CD₃Cl) δ 163.8, 156.2, 152.0, 137.5, 116.3, 108.2, 99.5, 81.2, 59.9, 37.1, 34.6, 28.6, 28.1, 25.3, 24.2, 14.1, −4.7, −5.6; Enantiometric excess was determined by HPLC with Chiralcel AD-H column (99.5:0.5 hexane:2-propanol), 0.5 mL/min; major enantiomer tᵣ=3.9 min, minor enantiomer tᵣ=4.6 min.

TABLE 8
Reaction of 2-methyl-6-nitroso-pyridine with acyclic dienes.

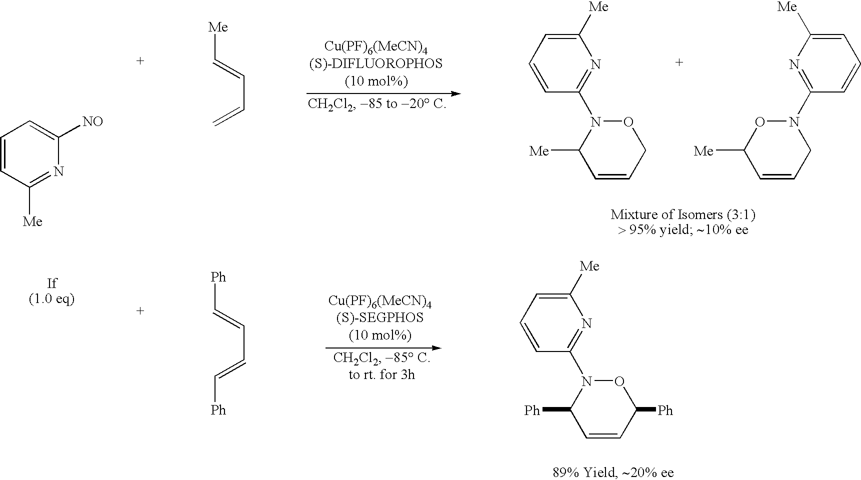

Table 8 further demonstrates the ability of this Diels-Alder reaction, as disclosed herein, to utilized acyclic diene substrates. The substrates shown in this table are only illustrative and are in no way limiting or exhaustive.

TABLE 9
Reaction of 2-methyl-6-nitroso-pyridine with
acyclic silyloxy-dienes.

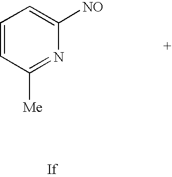

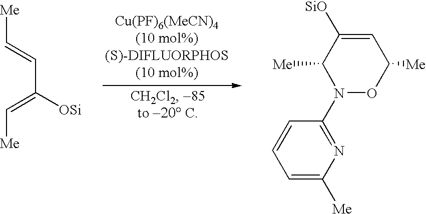

| | | |
|---|---|---|
| -OSi = -OTMS | XVII | 15% ee |
| -OSi = -OTBS | XVIII | 85% ee |
| -OSi = -OTIPS | XIX | >99% ee |

Table 9 demonstrates the ability of the Diels-Alder reaction, as disclosed herein, to utilize silyloxy-dienes.

### Example 18

### General Procedure for the Synthesis of XVIII

To a Schrenk tube was added Copper(I)(CH₃CN)₄ PF₆ (18.6 mg, 0.05 mmol) and (S)-(−) DIFLUOPHOS (35.8 mg, 0.0525 mmol). The mixture was dried under vacuum for 10 min, substituted with N₂ gas, and was added anhydrous CH₂Cl₂ (4 mL) and stirred for 1 h. The clear solution was then cooled to −85° C. and 1c dissolved in anhydrous CH₂Cl₂ (1 mL) was added dropwise. The resulting dark blue solution was stirred for 10 min, diene (0.6 mmol) dissolved in anhydrous CH₂Cl₂ was added dropwise in 1 h. The reaction mixture was gradually warmed to −20° C. in 5 h and was stirred at −20° C. for additional 1 h. The crude product was purified by silica gel chromatography to afford nitroso-Diels-Alder adduct XXVIII.

Characterization data from compound XVIII: Purification by flash column chromatography with elution by (95:5:0.02 hexane:ethylacetate:triethylamine) gave the product as colorless oil (56% yield, 85% ee); TLC R_{f} 0.7 (EtOAc/Hexanes, 1:9); [α]_{D}²⁸−185.4 (c=0.57, CHCl₃); FTIR (CD₃Cl) υₘₐₓ 2931, 2859, 1669, 1577, 1456, 1338, 1209 cm⁻¹; ¹H NMR (400 MHz, CD₃Cl) δ 7.44 (t, J=7.8 Hz, 1H), 6.90 (d, J=8.3 Hz, 1H), 6.58 (d, J=7.3 Hz, 1H), 4.65-4.77 (m, 3H), 2.41 (s, 3H), 1.24-1.29 (m, 6H), 0.95 (s, 9H), 0.21 (s, 3H), 0.19 (s, 3H); ¹³C NMR (125 MHz, CD₃Cl) δ 159.2, 156.6, 152.5, 137.7, 114.7, 106.2, 104.2, 71.7, 53.9, 25.6, 24.4, 20.0, 18.0, 14.3, −4.3, −4.8; MS (Cl) Exact Mass Calcd for C₁₂H₁₅N₂O (M+H)⁺: 203.1. Found: 203.1. Enantiometric excess was determined by HPLC with Chiralcel AD-H column (99.5:0.5 hexane:2-propanol), 0.5 mL/min; major enantiomer tᵣ=3.9 min, minor enantiomer tᵣ=4.3 min.

Characterization data for compound XIX: Purification by flash column chromatography with elution by (95:5:1 hexane:ethylacetate:TEA) provided as a colorless oil (95% yield, 81% ee); TLC R_{f} 0.8 (EtOAc/Hexanes, 1:5); [α]D ²⁵-103.9 (c=0.77, CHCl₃); FTIR (CD₃Cl) u max 2945, 2867, 1665, 1577, 1337, 1210, 1065 cm⁻¹; ¹H NMR (400 MHz, CD₃Cl) δ 7.35-7.48 (m, 6 H), 6.93 (d, J=8.3 Hz, 1H), 6.60 (d, J=7.4 Hz, 1H), 5.57 (s, 1H), 4.83-4.90 (m, 2H), 2.43 (s, 3H), 1.40 (d, J=6.5 Hz, 3H), 1.15-1.28 (m, 3H), 1.12 (s, 12H), 1.10 (s, 6H); ¹³C NMR (100 MHz, CD₃Cl) δ 159.3, 156.6, 153.3, 139.3, 137.8, 128.7, 128.5, 128.4, 115.0, 106.7, 101.1, 78.7, 54.5, 24.4, 18.0, 14.6, 12.6; MS (Cl) Exact Mass Calcd for C₂₆H₃₉N₂O₂Si (M+H)⁺: 439.3. Found: 439.1. Enantiometric excess was determined by HPLC with Chiralcel OD-H column (99.8:0.2 hexane:2-propanol), 0.5 mL/min; major enantiomer tᵣ=27.4 min, minor enantiomer tᵣ=19.4 min.

TABLE 10
Reaction of 2-methyl-6-nitroso-pyridine with a variety of
acyclic silyloxy-dienes.

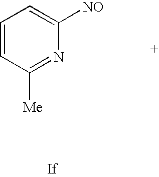

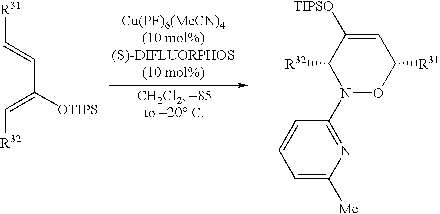

| Entry | Yield (D.R.)^{a}; % ee |
|---|---|
| 1 | 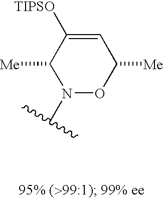 |
| 2 | 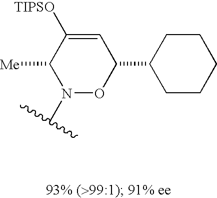 |
| 3 | 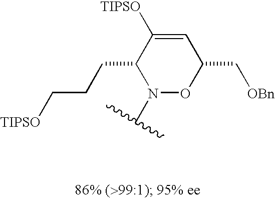 |
| 4 | 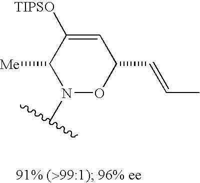 |
| 5 | 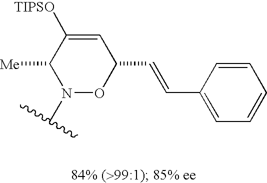 |
| 6 | 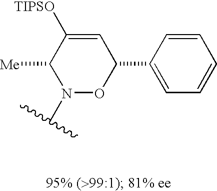 |
| 7 | 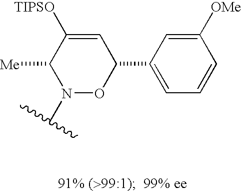 |
| 8 | 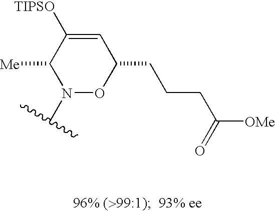 |
| 9 | 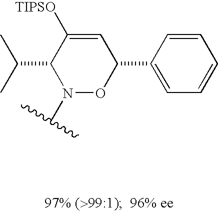 |
| 10 | 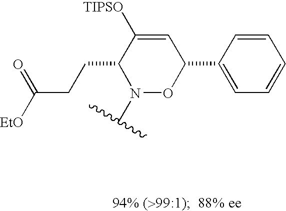 |
| 11 | 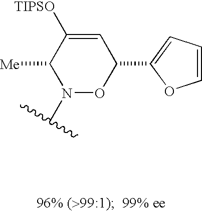 |

^{a}D.R. represents Diastereomeric Ratio

Table 10 demonstrates the ability of the Diels-Alder reaction, as disclosed herein, to utilize silyloxy-dienes in the presence of a variety of functional groups, including esters (entry 8) and alkenes (entry 4).

R³¹ and R³² are each independently selected from the group consisting of alkyl, cycloalkyl, alkoxy, alkylamino, alkylthio, aryl, arylalkyl, heterocyclyl, heteroaryl, halogen, silyloxy, carboxylic acid, ester, alkene, azide, amine, hydroxyl, imine, ketone, thiole, amide, silyl, nitrile, sulfoxide, sulfone, sulfonamide and nitroso.

Characterization data for Entry 6 of Table 10: Purification by flash column chromatography with elution by (95:5:1 hexane:ethylacetate:TEA) provided as a colorless oil (95% yield, 81% ee); TLC R_{f} 0.8 (EtOAc/Hexanes, 1:5); [α]_{D}²⁵−103.9 (c=0.77, CHCl₃); FTIR (CD₃Cl) υₘₐₓ 2945, 2867, 1665, 1577, 1337, 1210, 1065 cm⁻¹; ¹H NMR (400 MHz, CD₃Cl) δ 7.35-7.48 (m, 6H), 6.93 (d, J=8.3 Hz, 1H), 6.60 (d, J=7.4 Hz, 1H), 5.57 (s, 1H), 4.83-4.90 (m, 2H), 2.43 (s, 3H), 1.40 (d, J=6.5 Hz, 3H), 1.15-1.28 (m, 3H), 1.12 (s, 12H), 1.10 (s, 6H); ¹³C NMR (100 MHz, CD₃Cl) δ 159.3, 156.6, 153.3, 139.3, 137.8, 128.7, 128.5, 128.4, 115.0, 106.7, 101.1, 78.7, 54.5, 24.4, 18.0, 14.6, 12.6; MS (Cl) Exact Mass Calcd for C₂₆H₃₉N₂O₂Si (M+H)⁺: 439.3. Found: 439.1. Enantiometric excess was determined by HPLC with Chiralcel OD-H column (99.8:0.2 hexane:2-propanol), 0.5 mL/min; major enantiomer tᵣ=27.4 min, minor enantiomer tᵣ=19.4 min.

Characterization data for Entry 3 of Table 10: Purification by flash column chromatography with elution by (90:10:1 hexane:EtOAc:TEA) provided as a yellowish oil (86% yield, 95% ee). TLC R_{f} 0.8 (EtOAc/Hexane, 1:5); [α]_{D}²⁴−81.9° (c=0.29, CHCl₃); FTIR (CD₃Cl) υₘₐₓ 2947, 2866, 1671, 1590, 1577, 1452, 1254, 1211, 1096, 835 cm⁻¹; ¹H NMR (400 MHz, CD₃Cl) δ 7.42 (dd, J=8.2 Hz, J=7.4 Hz, 1H), 7.26-7.38 (m, 5H), 6.91 (d, J=8.3 Hz, 1H), 6.55 (d, J=7.3 Hz, 1H), 4.90-4.95 (m, 1H), 4.77-4.82 (m, 1H), 4.64 (dd, J=41.0 Hz, J=12.2 Hz 1H), 4.61 (d, J=1.3 Hz, 1H), 3.55-3.65 (m, 3H), 3.49 (dd, J=10.6 Hz, J=3.9 Hz 1H), 2.36 (s, 3H), 1.92-2.03 (m, 1H), 1.72-1.87 (m, 1H), 1.60-1.71 (m, 2H), 1.10-1.26 (m, 3H), 1.08 (d, J=2.7 Hz, 12H), 1.06 (d, J=2.6 Hz, 6H), 0.87 (s, 9H), 0.01 (s, 6H); ¹³C NMR (100 MHz, CD₃Cl) δ 159.0, 156.5, 153.3, 138.2, 137.7, 128.3, 127.6, 114.4, 106.0, 97.7, 73.4, 72.9, 72.3, 63.2, 56.3, 29.9, 27.5, 26.0, 24.3, 18.0, 12.6, −5.3; MS (Cl) Exact Mass Calcd for C₃₆H₆₁N₂O₄Si₂ (M+H)⁺: 641.4. Found: 641.3. Enantiometric excess was determined by HPLC with Chiralcel OD-H column (99.5:0.5 hexane:2-propanol), 1.0 mL/min; major enantiomer tᵣ=8.7 min, minor enantiomer tᵣ=6.6 min.

Characterization data for Entry 4 of Table 10: Purification by flash column chromatography with elution by (95:5 hexanes:ethylacetate) gave the product as colorless oil (91% yield, 96% ee); TLC R_{f} 0.8 (EtOAc/Hexanes, 1:5); [α]_{D}²⁷-117.7 (c=0.68, CHCl₃); FTIR (CD₃Cl) υₘₐₓ 2945, 2867, 1664, 1590, 157, 1454, 1340, 1208, 1065, 883 cm⁻¹; ¹H NMR (400 MHz, CD₃Cl) δ 7.36 (dd, J=8.2 Hz, J=7.5 Hz, 1H), 6.85 (d, J=8.3 Hz, 1H), 6.51 (d, J=7.3 Hz, 1H), 5.75-5.82 (m, 1H), 5.37-5.47 (m, 1H), 4.88 (br d J=7.8 Hz), 4.66-4.73 (m, 1H), 4.62 (d, J=0.9 Hz, 1H), 2.34 (s, 3H), 1.70 (dd, J=9.6 Hz, J=1.6 Hz, 3H), 1.23 (d, J=6.6 Hz, 3H), 1.15-1.20 (m, 3H), 1.05 (s, 12H), 1.08 (s, 6H); ¹³C NMR (100 MHz, CD₃Cl) δ 159.3, 156.6, 153.0, 137.7, 131.1, 129.2, 114.7, 106.4, 100.8, 76.7, 54.2, 24.4, 18.0, 17.9, 14.4, 12.6; MS (Cl) Exact Mass Calcd for C₂₃H₃₉N₂O₂Si (M+H)⁺: 403.3. Found: 403.1. Enantiometric excess was determined by HPLC with Chiralcel OD-H column (99.9:0.1 hexane:2-propanol), 0.5 mL/min; major enantiomer tᵣ=33.2 min, minor enantiomer tᵣ=11.7 min.

Characterization data for Entry 5 of Table 10: Purification by flash column chromatography with elution by (95:5:1 hexanes:ethylacetate:TEA) gave the product as colorless crystal (84% yield, 85% ee); TLC R_{f} 0.8 (EtOAc/Hexanes, 1:5); [α]_{D}²⁵−47.0 (c=0.90, CHCl₃); FTIR (CD₃Cl) υₘₐₓ 2944, 2867, 1665, 1578, 1456, 1337, 1211, 1122, 1066, 964, 884 cm⁻¹; ¹H NMR (500 MHz, CD₃Cl) δ 7.34-7.39 (m, 3H), 7.22-7.26 (m, 2H), 7.17-7.20 (m, 1H), 6.89 (d, J=8.3 Hz, 1H), 6.62 (d, J=15.9 Hz, 1H), 6.52 (d, J=7.4 Hz, 1H), 6.09 (dd, J=15.9 Hz, J=7.7 Hz, 1H), 5.09-5.12 (m, 1H), 4.72-4.78 (m, 1H), 4.69 (br d J=1.1 Hz), 2.34 (s, 3H), 1.27 (d, J=6.6 Hz, 3H), 1.08-1.20 (m, 3H), 1.04 (d, J=2.2 Hz, 12H), 1.03 (d, J=2.1 Hz, 6H),; ¹³C NMR (100 MHz, CD₃Cl) δ 159.3, 156.6, 153.4, 137.8, 136.4, 133.7, 128.6, 128.0, 127.1, 126.7, 114.9, 106.5, 100.3, 76.9, 54.4, 24.4, 18.0, 14.4, 12.6; MS (Cl) Exact Mass Calcd for C₂₃H₃₉N₂O₂Si (M+H)⁺: 465.3. Found: 465.1. Enantiometric excess was determined by HPLC with Chiralcel OD-H column (99.9:0.1 hexane:2-propanol), 0.5 mL/min; major enantiomer tᵣ=30.6 min, minor enantiomer tᵣ=25.7 min.

Characterization data for Entry 11 of Table 10: Purification by flash column chromatography with elution by (95:5:hexane:ethylacetate) provided as a colorless oil (91% yield, 95% ee); TLC R_{f} 0.8 (EtOAc/Hexanes, 1:5); [α]_{D}²⁵−120.6 (c=0.82, CHCl₃); FTIR (CD₃Cl) υₘₐₓ 2945, 2867, 1668, 1590, 1577, 1454, 1338, 1211, 1065, 833 cm⁻¹; ¹H NMR (500 MHz, CD₃Cl) δ 7.43-7.48 (m, 2H), 6.99 (d, J=8.3 Hz, 1H), 6.62 (d, J=7.4 Hz, 1H), 6.37-6.42 (m, 2H), 5.64-5.67 (m, 1H), 4.90 (d, J=1.4 Hz, 1H), 4.81-4.86 (m, 1H), 2.43 (s, 3H), 1.34 (d, J=6.5 Hz, 3H), 1.21-1.30 (m, 3H), 1.14 (d, J=5.7 Hz, 12H), 1.12 (d, J=5.8 Hz, 6H); ¹³C NMR (125 MHz, CD₃Cl) δ 159.1, 156.6, 154.5, 152.7, 143.0, 137.8, 115.1, 110.4, 109.2, 106.7, 97.8, 71.3, 54.7, 24.4, 18.0, 14.2, 12.6; MS (Cl) Exact Mass Calcd for C₂₄H₃₇N₂O₃Si (M+H)⁺: 429.3. Found: 429.1. Enantiometric excess was determined by HPLC with Chiralcel OD-H column (99.5:0.5 hexane:2-propanol), 0.5 mL/min; major enantiomer tᵣ=10.4 min, minor enantiomer tᵣ=8.5 min.

Characterization data for Entry 9 of Table 10: Purification by flash column chromatography with elution by (95:5:hexane:ethylacetate) provided as a colorless oil (97% yield, 96% ee); TLC R_{f} 0.8 (EtOAc/Hexanes, 1:5); [α]_{D}²⁶−143.6 (c=0.57, CHCl₃); FTIR (CD₃Cl) υₘₐₓ 2929, 2866, 1668, 1590, 1453, 1339, 1210, 882 cm⁻¹; ¹H NMR (400 MHz, CD₃Cl) δ 7.36-7.48 (m, 6H), 6.95 (d, J=8.3 Hz, 1H), 6.56 (d, J=7.2 Hz, 1H), 5.45-5.47 (m, 1H), 5.02-5.04 (m, 1H), 4.84 (br s, 1H), 2.38-2.43 (m, 1H), 2.38 (s, 3H), 1.06-1.25 (m, 27H); ¹³C NMR (100 MHz, CD₃Cl) δ 159.4, 156.7, 151.3, 139.1, 137.7, 128.7, 128.6, 128.5, 114.0, 105.7, 101.5, 75.1, 59.9, 30.4, 24.4, 20.1, 19.8, 18.1, 18.0, 12.6; MS (Cl) Exact Mass Calcd for C₂₈H₄₃N₂O₂Si (M+H)⁺: 467.3. Found: 467.2. Enantiometric excess was determined by HPLC with Chiralcel OD-H column (99.8:0.2 hexane:2-propanol), 0.5 mL/min; major enantiomer tᵣ=13.3 min, minor enantiomer tᵣ=11.8 min.

Characterization data for Entry 2 of Table 10: Purification by flash column chromatography with elution by (95:5:hexane:ethylacetate) provided as a colorless oil (93% yield, 91% ee); TLC R_{f} 0.8 (EtOAc/Hexanes, 1:5); [α]_{D}²⁶−116.8 (c=0.69, CHCl₃); FTIR (CD₃Cl) υₘₐₓ 2945, 2867, 1667, 1588, 1577, 1449, 1311, 1211, 1195 cm⁻¹; ¹H NMR (400 MHz, CD₃Cl) δ 7.43 (dd, J=8.1 Hz, J=7.5 Hz, 1H), 6.87 (d, J=8.3 Hz, 1H), 6.57 (d, J=7.2 Hz, 1H), 4.74-4.77 (m, 1H), 4.71 (br s, 1H), 4.37 (br d, J=5.0 Hz, 1H), 2.40 (s, 3H), 1.43-1.91 (m, 6H), 1.16-1.29 (m, 11H), 1.11 (s, 12H), 1.10 (s, 6H); ¹³C NMR (100 MHz, CD₃Cl) δ 159.4, 156.5, 153.0, 137.6, 114.5, 106.3, 99.6, 79.4, 54.2, 41.8, 28.8, 27.9, 26.5, 26.3, 26.2, 24.4 18.0, 14.6, 12.6; MS (Cl) Exact Mass Calcd for C₂₆H₄₅N₂O₂Si (M+H)⁺: 445.3. Found: 445.2. Enantiometric excess was determined by HPLC with Chiralcel OD-H column (99.6:0.4 hexane:2-propanol), 0.5 mL/min; major enantiomer tᵣ=7.8 min, minor enantiomer tᵣ=7.0 min.

Characterization data for Entry 10 of Table 10: Purification by flash column chromatography with elution by (95:5:hexane:ethylacetate) provided as a colorless oil (94% yield, 88% ee); TLC R_{f} 0.8 (EtOAc/Hexanes, 1:5); [α]_{D}²⁶ −101.8 (c=0.66, CHCl₃); FTIR (CD₃Cl) υₘₐₓ 2945, 2867, 1735, 1669, 1589, 1576, 1455, 1212 cm⁻¹; ¹H NMR (400 MHz, CD₃Cl) δ 7.35-7.49 (m, 6H), 6.95 (d, J=8.3 Hz, 1H), 6.59 (d, J=7.3 Hz, 1H), 5.48-5.51 (m, 1H), 5.04-5.10 (m, 1H), 4.82 (br s, 1H), 4.09 (q, J=7.1 Hz, 2H), 2.51-2.58 (m, 2H), 2.39 (s, 3H), 2.27-2.37 (m, 2H), 1.16 (d, J=5.4 Hz, 12H), 1.08 (d, J=5.4 Hz, 6H); ¹³C NMR (100 MHz, CD₃Cl) δ 173.7, 158.8, 156.7, 151.6, 138.8, 137.8, 128.8, 128.6, 128.4, 114.6, 106.0, 101.2, 76.0, 60.1, 55.3, 31.6, 26.6, 24.3, 18.0, 14.2, 12.5; MS (Cl) Exact Mass Calcd for C₃₀H₄₅N₂O₄Si (M+H)⁺: 525.3. Found: 525.2. Enantiometric excess was determined by HPLC with Chiralcel OD-H column (99:1 hexane:2-propanol), 0.5 mL/min; major enantiomer tᵣ=12.2 min, minor enantiomer tᵣ=10.0 min.

Characterization data for Entry 8 of Table 10: Purification by flash column chromatography with elution by (95:5:hexane:ethylacetate) provided as a colorless oil (96% yield, 93% ee); TLC R_{f} 0.8 (EtOAc/Hexanes, 1:5); [α]_{D}²⁶-149.1 (c=0.85, CHCl₃); FTIR (CD₃Cl) υₘₐₓ 2945, 2867, 1742, 1669, 1590, 1576, 1454, 1337, 1237, 1166, 883, 785, 685 cm⁻¹; ¹H NMR (500 MHz, CD₃Cl) δ 7.44 (dd, J=8.1 Hz, J=7.5 Hz, 1H), 6.87 (d, J=8.3 Hz, 1H), 6.59 (d, J=7.3 Hz, 1H), 4.74 (q, J=6.5 Hz, 1H), 4.68 (br s, 1H), 4.58 (br t, J=6.0 Hz, 1H), 3.67 (s, 3H), 2.40 (s, 3H), 2.38-2.40 (m, 2H), 1.78-1.94 (m, 2H), 1.59-1.63 (m, 2H), 1.26 (d, J=6.5 Hz, 3H), 1.19-1.24 (m, 3H) 1.13 (s, 12H), 1.10 (s, 6H); ¹³C NMR (125 MHz, CD₃Cl) δ 173.9, 159.3, 156.6, 153.0, 137.7, 114.8, 106.2, 101.2, 75.4, 54.5, 51.5, 34.0, 33.9, 24.4, 20.7, 18.0, 14.3, 12.6; MS (Cl) Exact Mass Calcd for C₂₅H₄₃N₂O₄Si (M+H)⁺: 463.3. Found: 463.2. Enantiometric excess was determined by HPLC with Chiralcel OD-H column (99:1 hexane:2-propanol), 0.5 mL/min; major enantiomer tᵣ=10.0 min, minor enantiomer tᵣ=8.8 min.

Characterization data for Entry 7 of Table 10: Purification by flash column chromatography with elution by (95:5:hexane:ethylacetate) provided as a colorless oil (91% yield, 99% ee); TLC R_{f} 0.8 (EtOAc/Hexanes, 1:5); [α]_{D}²⁶-106.3 (c=0.57, CHCl₃); FTIR (CD₃Cl) υₘₐₓ 2945, 2867, 1665, 1589, 1454, 1210, 882 cm⁻¹; ¹H NMR (500 MHz, CD₃Cl) δ 7.43 (dd, J=8.1 Hz, J=7.5 Hz, 1H), 7.31 (dd, J=7.9 Hz, J=7.8 Hz, 1H), 7.04 (d, J=7.6 Hz, 1H), 7.01 (s, 1H), 6.89-6.94 (m, 2H), 6.60 (d, J=7.3 Hz, 1H), 4.87 (q, J=6.5 Hz, 1H), 4.83 (s, 1H), 3.83 (s, 3H), 2.43 (s, 3H), 1.40 (d, J=6.5 Hz, 1H), 1.19-1.27 (m, 3H), 1.12 (d, J=2.5 Hz, 12H), 1.10 (d, J=2.5 Hz, 6H); ¹³C NMR (125 MHz, CD₃Cl) δ 159.7, 159.2, 156.6, 153.3, 140.9, 137.8, 129.5, 120.8, 115.0, 114.5, 113.5, 106.7, 100.9, 78.6, 55.2, 54.5, 24.4, 18.0, 14.6, 12.6; MS (Cl) Exact Mass Calcd for C₂₅H₄₃N₂O₄Si (M+H)⁺: 469.3. Found: 469.1. Enantiometric excess was determined by HPLC with Chiralcel OD-H column (99.8:0.2 hexane:2-propanol), 0.5 mL/min; major enantiomer tᵣ=34.7 min, minor enantiomer tᵣ=25.8 min.

TABLE 11
Reaction of 2-methyl-6-nitroso-pyridine with a variety of cyclic
silyloxy-dienes.

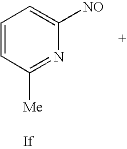

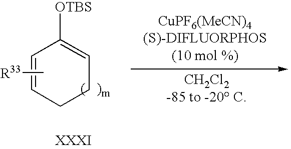

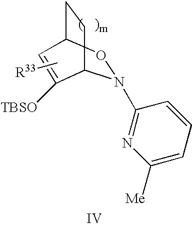

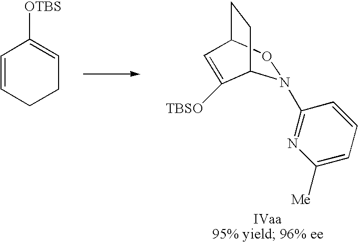

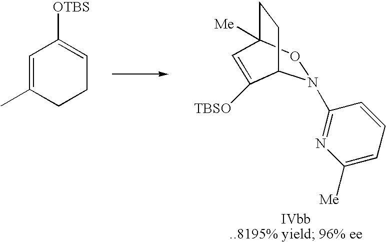

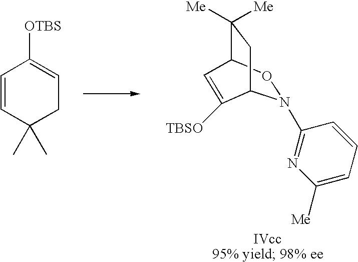

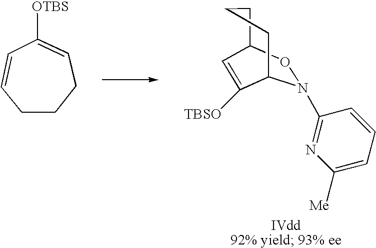

Table 11 demonstrates the ability of the Diels-Alder reaction, as disclosed herein, to utilize cyclic silyloxy-dienes.

R³³ represents 0 to 4 substituents each of which is independently selected from the group consisting of alkyl, cycloalkyl, alkoxy, alkylamino, alkylthio, aryl, arylalkyl, heterocyclyl, heteroaryl, halogen, silyloxy, carboxylic acid, ester, alkene, azide, amine, hydroxyl, imine, ketone, thiole, amide, silyl, nitrile, sulfoxide, sulfone, sulfonamide and nitroso.
m is 0, 1, or 2.

Characterization data for compound IVcc: purification by flash column chromatography with elution by (9:1:0.02 hexane:ethylacetate:triethylamine) gave the product as colorless oil (95% yield, 98% ee); TLC R_{f} 0.8 (EtOAc/Hexanes, 1:5); [α]_{D}²⁶−80.9 (c=0.92, CHCl₃); R_{f} 0.7 (EtOAc/Hexanes, 1:5); FTIR (CD₃Cl) υₘₐₓ 2953, 2859, 1639, 1577, 1450, 1363, 1252, 1222 cm⁻¹; ¹H NMR (400 MHz, CD₃Cl) δ 7.61 (dd, J=7.9 Hz, J=7.7 Hz, 1H), 7.00 (d, J=8.2 Hz, 1H), 6.80 (d, J=7.4 Hz, 1H), 5.35 (dd, J=6.6 Hz, J=2.2 Hz, 1H), 5.15-5.21 (m, 1H), 4.43 (d, J=6.6 Hz, 1H), 2.61 (s, 3H), 2.15 (dd, J=12.9 Hz, J=3.3 Hz, 1H), 1.75 (dd, J=13.0 Hz, J=3.0 Hz, 1H), 1.51 (s, 3H), 1.12 (s, 3H), 0.99 (s, 9H), 0.26 (s, 3H), 0.24 (s, 3H); ¹³C NMR (100 MHz, CD₃Cl) δ 163.8, 156.2, 152.0, 137.5, 116.3, 108.2, 99.5, 81.2, 60.0, 37.1, 34.6, 28.6, 28.1, 25.3, 24.2, 14.1, −4.7, −5.6; MS (Cl) Exact Mass Calcd for C₂₀H₃₃N₂O₂Si (M+H)⁺: 361.2. Found: 361.1. Enantiometric excess was determined by HPLC with Chiralcel AD-H column (99.5:0.5 hexane:2-propanol), 1.0 mL/min; major enantiomer tᵣ=3.7 min, minor enantiomer tᵣ=4.1 min.

Characterization data for compound IVdd: Purification by flash column chromatography with elution by (9:1:0.02 hexane:ethylacetate:triethylamine) gave the product as colorless oil (95% yield, 93% ee); TLC R_{f} 0.8 (EtOAc/Hexanes, 1:5); [α]_{D}²⁶ −11.4 (c=1.53, CHCl₃); FTIR (CD₃Cl) υₘₐₓ 2930, 2858, 1650, 1589, 1576, 1450, 1253, 1225, 888 cm⁻¹; ¹H NMR (400 MHz, CD₃Cl) δ 7.40 (dd, J=8.0 Hz, J=7.7 Hz, 1H), 6.82 (d, J=8.2 Hz, 1H), 6.60 (d, J=7.4 Hz, 1H), 5.13 (dd, J=7.9 Hz, J=2.2 Hz, 1H), 4.85-4.93 (m, 1H), 4.74 (dd, J=7.0 Hz, J=2.6 Hz, 1H), 2.34 (s, 3H), 2.09-2.21 (m, 1H), 1.83-1.93 (m, 2H), 1.55-1.72 (m, 2H), 1.38-1.53 (m, 1H), 0.81 (s, 9H), 0.02 (s, 3H), −0.2 (s, 3H); ¹³C NMR (100 MHz, CD₃Cl) δ 163.7, 156.2, 153.0, 137.6, 115.7, 107.8, 95.6, 74.1, 63.1, 33.1, 25.9, 25.3, 24.2, 18.7, −4.7, −5.6; Enantiometric excess was determined by HPLC with Chiralcel OD-H column (96:4 hexane:2-propanol), 1.0 mL/min; major enantiomer tᵣ=7.0 min, minor enantiomer tᵣ=5.3 min.

Characterization data for compound Ivbb: Purification by flash column chromatography with elution by (9:1:0.02 hexane:ethylacetate:triethylamine) provided as a white crystal (95% yield, 99% ee); TLC R_{f} 0.7 (EtOAc/Hexanes/triethyamine, 1:5:0.02); [α]_{D}²⁸-113.5° (c=0.40, CHCl₃); FTIR (CD₃Cl) υₘₐₓ 2931, 2858, 1653, 1575, 1473, 1254, 1229 cm⁻¹; ¹H NMR (400 MHz, CD₃Cl) δ 7.39 (t, J=7.8 Hz, 1H), 6.77 (d, J=8.2 Hz, 1H), 6.62 (d, J=7.4 Hz, 1H), 5.03-5.07 (m, 1H), 4.91 (br d, J=2.5 Hz, 1H), 5.05-5.09 (m, 1H), 2.39 (s, 3H), 2.22-2.26 (m, 1H), 1.92-1.99 (m, 1H), 1.74-1.81 (m, 1H), 1.52 (s, 3H), 1.40-1.48 (m, 1H), 0.78 (s, 9H), 0.02 (s, 3H), −0.26 (s, 3H); ¹³C NMR (100 MHz, CD₃Cl) δ 164.1, 156.2, 153.4, 137.5, 116.1, 108.0, 104.2, 77.4, 58.6, 32.7, 25.3, 24.2, 23.6, 22.1, 17.7, −4.6, −5.8. MS (Cl) Exact Mass Calcd for C₁₉H₃₁N₂O₂Si (M+H)⁺: 347.2. Found: 347.1. Enantiometric excess was determined by HPLC with Chiralcel OD-H column (97.5:2.5 hexane:2-propanol), 1.0 mL/min; major enantiomer tᵣ=7.2 min, minor enantiomer tᵣ=4.8 min.

### Example 19

### Dihydroxylation of Compound XX

The following scheme demonstrates different methods that may be used to functionalize the products resulting from the Diels-Alder reaction, as disclosed herein.

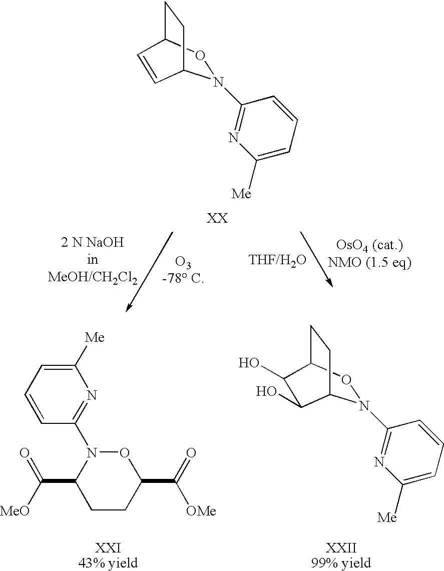

The following scheme demonstrates how the pyridine can be cleaved from the Diels-Alder product and how the cyclic hydroxylamine can be cleaved.

To the solution of Nitroso Diels-Alder (10 mmol) adduct XX in THF/H₂O (15/1, 30 mL) was added OsO₄ (2 mL, 2 wt % in H₂O) and cooled to −20° C. The resulting solution was added 4-Methylmorpholine N-oxide (15 mmol) and was allowed to warm to r.t. The solution was added Et₂O and sat. aq. Na₂S₂O₃. Aqueous layer was discarded and the organic layer was washed with sat. aq. NH₄Cl and sat. aq. NaCl and dried over Na2SO4 and concentrated under reduced pressure. The residue was purified by silica gel chromatography to give a colorless crystal XXII.

Characterization data for compound XXII: ¹H NMR (400 MHz, CD₃Cl) δ 7.52 (dd, J=8.2 Hz, J=7.4 Hz, 1H), 7.07 (d, J=8.3 Hz, 1H), 6.68 (d, J=7.3 Hz, 1H), 4.67 (br s, 1H), 4.53-4.57 (m, 1H), 4.44-4.48 (m, 1H), 4.20-4.24 (m, 2H), 3.31 (br s, 1H), 2.43 (s, 3H), 1.95-2.17 (m, 2H), 1.68-1.82 (m, 2H).

### Example 20

### Ozonolysis of Compound XX

To the Diels-Alder adduct (2 mmol) in CH₂Cl₂ (10 mL) was added 2.5 N NaOH in MeOH (10 mL). O₃ was bubbled through the solution for 5 h. The solution was bubbled with N₂ and was concentrated under reduced pressure. The organic was extracted with Et₂O and washed with H₂O and sat. aq. NH₄Cl and sat. aq. NaCl and dried over Na₂SO₄ and concentrated under reduced pressure. The residue was purified by silica gel chromatography to give a colorless crystal XXI.

Characterization data for compound XXI: ¹H NMR (400 MHz, CD₃Cl) δ 7.51 (dd, J=8.2 Hz, J=7.4 Hz, 1H), 7.04 (d, J=8.3 Hz, 1H), 6.66 (d, J=7.3 Hz, 1H), 5.39-5.42 (m, 1H), 4.51 (dd, J=11.0 Hz, J=3.0 Hz, 1H), 3.80 (s, 3H), 3.68 (s, 3H), 2.43-2.50 (m, 1H), 2.39 (s, 3H), 1.80-2.15 (m, 1H).

In the following scheme the pyridine and the N—O bond of the Diels-Alder product XVIII are cleaved.

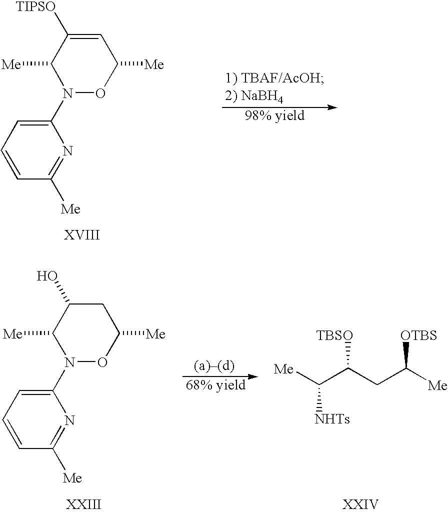

To a solution of XVIII (1.50 g, 4.0 mmol) in THF (30 mL) was added AcOH (264 mg, 4.4 mmol). The mixture was cooled with CO₂/Acetone bath. TBAF (1.0M in THF, 4.4 mL, 4.4 mmol) was added dropwise to the solution and the resulting mixture was allowed to warm to room temperature by removing the cooling bath. Sat. aq. NH₄Cl (15 mL) was added and the organic was extracted with Et₂O (30 mL). Organic layer was washed by sat. aq. NaHCO₂ (15 mL), sat. aq. NaCl (15 mL) and dried over Na₂SO₄ and concentrated under reduced pressure. The residue was added MeOH (20 mL) and cooled with ice/water bath. NaBH₄ (166 mg 4.4 mmol) was added and was stirred at same temperature for 2 h. The mixture was concentrated under reduced pressure and extracted with Et₂O (40 mL). Organic layer was then washed with sat. aq. NH₄Cl (20 mL), sat. aq. NaHCO₃ (20 mL), sat. aq. NaCl (20 mL) and dried over Na₂SO₄ and concentrated under reduced pressure. The residue was purified by silica gel chromatography to give XXIII as a colorless oil (871 mg, 3.9 mmol, 98% yield in 2 steps).

Characterization data for compound XXIII: TLC R_{f} 0.6 (EtOAc/Hexanes, 1:2); [α]_{D}²⁶−107.4 (c=0.76, CHCl₃); FTIR (CD₃Cl) υₘₐₓ 3384, 2975, 2939, 1578, 1452, 1375, 1337, 1149, 1100, 1053, 785 cm⁻¹; ¹H NMR (500 MHz, CD₃Cl) δ 7.44 (dd, J=8.0 Hz, J=7.7 Hz, 1H), 6.88 (d, J=8.3 Hz, 1H), 6.60 (d, J=7.4 Hz, 1H), 4.82-4.86 (m, 1H), 4.19-4.23 (m, 1H), 3.97-4.02 (m, 1H), 2.41 (s, 3H), 1.80-1.86 (m, 1H), 1.64 (dd, J=24.0 Hz, J=11.5 Hz, 1H), 1.30 (d, J=6.3 Hz, 3H), 1.10 (d, J=6.7 Hz, 3H); ¹³C NMR (125 MHz, CD₃Cl) δ 159.7, 156.6, 138.0, 115.0, 106.5, 74.3, 74.2, 67.4, 55.1, 36.7, 24.3, 20.0, 6.8; MS (Cl) Exact Mass Calcd for C₁₂H₁₉N₂O₂ (M+H)⁺: 223.1. Found: 223.1.

Step (a): To a solution of XXIII (777 mg, 3.5 mmol) in MeOH (15 mL), 10% (dry basis) wet Pd/C (78 mg) basis, ACOH (264 mg, 4.4 mmol). The flask was substituted by H₂ gas (×3) and warmed to 45° C. and stirred vigorously at same temperature for 3 h. The mixture was cooled to RT and filtered through a short pad of Celite, concentrated under reduced pressure. The residue was added 2,2-dimethoxypropane (15 mL), TsOH—H₂O (1.9 mg 0.01 mmol) and the mixture was stirred at 80° C. for 2 h, and concentrated under reduced pressure. The organic was extracted with Et₂O (15 mL) and washed with aq. NaHCO₃ (15 mL), sat. aq. NaCl (15 mL) and dried over Na₂SO₄ and concentrated under reduced pressure. The residue was used for next reaction without further purification.

Step (b): The obtained residue was dissolved in 1,2-dichloroethane (10 mL) and was added N,N′-diisopropylethylamine (3.6 mL, 21 mmol) and Ts₂O (3.4 g, 10.5 mmol). The mixture was stirred at reflux (bath temp. 100° C.) for 24 h. The reaction mixture was cooled to r.t. and was added CH₂Cl₂ (30 mL). The organic layer was washed with sat. aq. NH₄Cl (10 mL), sat. aq. NaHCO₃ (10 mL), sat. aq. NaCl (10 mL) and dried over Na₂SO₄ and concentrated under reduced pressure. The residue was purified by silica gel chromatography to give the tosylate as a brownish oil (1.17 g, 2.8 mmol, 80% yield in 2 steps).

Step (c): To the obtained residue (418 mg, 1.0 mmol) in MeOH (10 mL) was added TsOH—H₂O (1.9 mg 0.01 mmol) and the mixture was stirred at 60° C. for 2 h. The reaction mixture was concentrated under reduced pressure and extracted with Et₂O (15 mL). The organic layer was washed with NH₄Cl (10 mL), sat. aq. NaHCO₃ (10 mL), sat. aq. NaCl (10 mL) and dried over Na₂SO₄ and concentrated under reduced pressure. The obtained residue was dissolved in CH₂Cl₂ (10 mL) and was cooled to 0° C. The mixture was added 2,6-Lutidine (0.51 mL, 4.4 mmol), TBSOTf (0.51 mL, 2.2 mmol) and was stirred at room temperature for 3 h. The reaction mixture was added sat. aq. NaHCO₃ (10 mL) and extracted with CH₂Cl₂ (10 mL). Organic layer was washed with sat. aq. NaCl (10 mL) and dried over Na₂SO₄ and concentrated under reduced pressure. The residue was purified by silica gel chromatography to give DiTBS protected alcohol as a colorless oil (576 mg, 0.95 mmol, 95% yield in 2 steps).

Step (d): To the solution of the obtained residue (485 mg. 0.8 mmol) in CH₂Cl₂ (10 mL) was added MeOTf (144 mg, 0.88 mmol) at 0° C. The reaction mixture was allowed to warm to r.t. and stirred for additional 12 h. Sat. aq. Na₂CO₃ (10 mL), was added and stirred vigorously for 15 min. The organic layer was washed with sat. aq. NaCl (10 mL) and dried over Na₂SO₄ and concentrated under reduced pressure. The residue was added MeOH (5 mL), 10N aq. KOH (10 mL) and stirred at 60° C. for 2 h. The mixture was concentrated under reduced pressure and the organic was extracted by Et₂O (10 mL). The organic was washed with NH₄Cl (10 mL×2), sat. aq. NaHCO₃ (10 mL), sat. aq. NaCl (10 mL) and dried over Na₂SO₄ and concentrated under reduced pressure. The residue was purified by silica gel chromatography to give 7 as a white solid. (371 mg, 0.72 mmol, 90%, 2 steps)

Characterization data for compound XXIV: FTIR (CD₃Cl) υₘₐₓ 3276, 2929, 2856, 1472, 1331, 1256, 1162, 1074, 835 cm⁻¹; ¹H NMR (500 MHz, CD₃Cl) δ 7.76 (d, J=6.5 Hz, 2H), 7.28 (d, J=7.9 Hz, 2H), 4.62 (d, J=8.9 Hz, 1H), 3.85-3.88 (m, 1H), 3.57-3.61 (m, 1H), 3.37-3.41 (m, 1H), 2.41 (s, 3H), 1.52-1.56 (m, 1H), 1.26-1.32 (m, 1H), 0.99 (d, J=6.3 Hz, 6H), 0.88 (s, 9H), 0.86 (s, 9H), 0.05 (s, 3H), 0.04 (s, 3H), 0.03 (s, 3H), 0.02 (s, 3H); ¹³C NMR (125 MHz, CD₃Cl) δ 173.7, 158.8, 156.7, 151.6, 138.8, 137.8, 128.8, 128.6, 128.4, 114.6, 106.0, 101.2, 76.0, 60.1, 55.3, 31.6, 26.6, 24.3, 18.0, 14.2, 12.5.

TABLE 12
Competitive Reaction

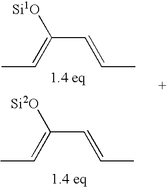

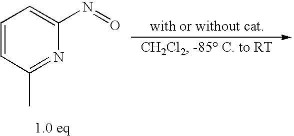

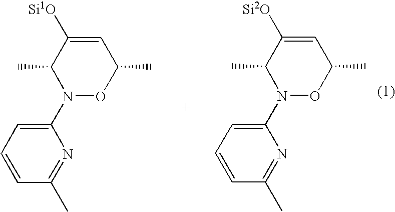

| | |
|---|---|
| Si¹ = TIPS, Si² = TMS | |
| no catalysis | >99:<1 |
| with CuPF₆(MeCN)₄-DIFLUORPHOS | >99 (99% ee):<1 |
| Si¹ = TIPS, Si² = TBS | |
| no catalysis | 3:1 |
| with CuPF₆(MeCN)₄-DIFLUORPHOS | 11(99% ee):1 |

Without catalysis: To the solution of mixture of silyloxydienes (0.7 mmol each) in CH₂Cl₂ (4 mL) was added dropwise 6-Methyl-2-nitrosopyridine (0.5 mmol) in CH₂Cl₂ (2 mL) at −85° C. The reaction mixture was allowed to warm to r.t. in 5 h and stirred for additional 1 h. The mixture was concentrated under reduced pressure and purified through SiO₂ column. TIPS vs TMS: No product from TMSoxydiene was obtained and 3c was obtained (179 mg, 0.47 mmol). TIPS vs TBS: products were unable to separate through SiO₂ column. The mixture of the products were collected (154 mg) and ratio (3:1) was determined by ¹H NMR.

With catalysis: To a Schrenk tube was added Copper(I)(CH₃CN)₄ PF₆ (18.6 mg, 0.05 mmol) and (S)-(−) DIFLUOPHOS (35.8 mg, 0.0525 mmol). The mixture was dried under vacuum for 10 min, substituted with N₂ gas, and anhydrous CH₂Cl₂ (4 mL) and stirred for 1 h. The clear solution was then cooled to −85° C. and was added mixture of silyloxydienes (0.7 mmol each) in CH₂Cl₂ (1 mL). The mixture was added dropwise 1c (0.5 mmol) dissolved in anhydrous CH₂Cl₂ (1 mL) in 1 h and stirried at the same temperature for 1 h. The reaction mixture was gradually warmed to −20° C. in 5 h and was stirred at −20° C. for additional 1 h. The crude product was purified by silica gel chromatography. TIPS vs. TMS: No product from TMSoxydiene was obtained and 3c was obtained (179 mg, 0.47 mmol, 99% ee). TIPS vs. TBS: products were unable to separate through SiO₂ column. The mixture of the products were collected (162 mg, 99% ee for 3c) and ratio (11:1) was determined by ¹H NMR.

TABLE 13
Competitive Reaction No. 2.

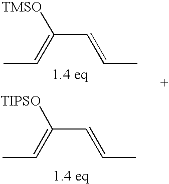

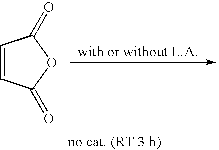

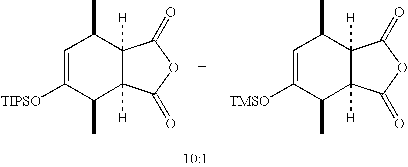

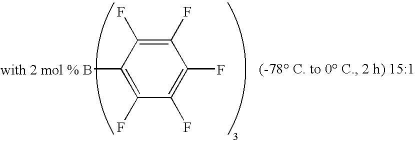

Without catalysis: To the solution of mixture of silyloxydienes (0.7 mmol each) in CH₂Cl₂ (4 mL) was added dropwise maleic anhydryde (0.5 mmol) in CH₂Cl₂ (2 mL) at 0° C. The reaction mixture was allowed to warm to r.t. and was stirred for 3 h. The mixture was concentrated under reduced pressure and purified through short pad of SiO₂ column treated with 5% TEA in Hexane. The mixture of two products was obtained (160 mg). The ratio (10:1) was determined by ¹H NMR.

With catalysis: To the solution of mixture of silyloxydienes (0.7 mmol each) in CH₂Cl₂ (4 mL) was added tris(pentafluorophenyl)borane (0.01 mmol) at −78° C. The mixture was added dropwise maleic anhydryde (0.5 mmol) at same temperature. The reaction mixture was allowed to warm to 0° C. in 2 h. The mixture was concentrated under reduced pressure and purified through short pad of SiO₂ column treated with 5% TEA in Hexane. The mixture of two products was obtained (168 mg). The ratio (15:1) was determined by ¹H NMR.

Characterization data for 4,7-Dimethyl-5-triisopropylsilanyloxy-3a,4,7,7a-tetrahydro-isobenzofuran-1,3-dione: FTIR (CD₃Cl) υₘₐₓ 1854, 1773, 1664, 1458, 1347, 1295, 1209, 1088, 1068, 1018, 933, 883, 850, 714 cm⁻¹; ¹H NMR (500 MHz, CD₃Cl) δ 4.58 (dd, J=3.4 Hz, J=2.5 Hz, 1H), 3.27 (dd, J=9.2 Hz, J=6.1 Hz, 1H), 3.18 (dd, J=9.2 Hz, J=6.1 Hz, 1H), 2.59-2.63 (m, 1H), 2.44-2.48 (m, 1H), 1.41 (d, J=7.3 Hz, 3H), 1.37 (d, J=7.3 Hz, 1H), 1.13-1.20 (m, 3H), 1.04 (d, J=2.7 Hz, 12H), 1.02 (d, J=2.6 Hz, 6H); ¹³C NMR (125 MHz, CD₃Cl) δ 171.6, 171.4, 153.4, 103.1, 47.0, 46.6, 34.1, 30.5, 17.9, 17.8, 17.2, 12.5.

Although the invention herein has been described in connection with a preferred embodiment thereof, it will be appreciated by those skilled in the art that additions, modifications, substitutions, and deletions not specifically described may be made without departing from the spirit and scope of the invention as defined in the appended claims.

It is intended that the foregoing detailed description be regarded as illustrative rather than limiting, and that it be understood that it is the following claims, including all equivalents, that are intended to define the spirit and scope of this invention.

1. A process of enantioselective chemical synthesis, comprising, reacting a C-nitroso dienophile and a 1,3-diene in the presence of a catalytic amount of an asymmetric bidentate ligand and a metal, to produce an enantiomerically enriched cycloadduct.

2. The process of claim 1, where the C-nitroso dienophile is an aromatic C-nitroso dienophile, in which there is a bond between a nitrogen of the nitroso group and a carbon of the aromatic ring.

3. The process of claim 2, where the aromatic C-nitroso dienophile is a compound of formula (I): 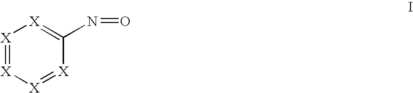
where:
each X is independently selected from the group consisting of —CR¹— or —N—;
R¹ is independently selected from the group consisting of hydrogen, alkyl, cycloalkyl, alkoxy, alkylamino, alkylthio, halogen, heterocyclyl, aryl, heteroaryl, arylalkyl, and O-silyl.

4. The process of claim 3, where the C-nitroso dienophile is a compound of formula (Ib): 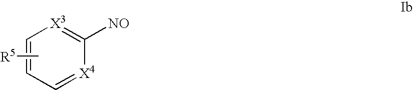
where:
X³ and X⁴ are independently selected from the group consisting of —CR⁴— and —N—; and
R⁴ is independently selected from the group consisting of hydrogen, alkyl, cycloalkyl, alkoxy, alkylamino, alkylthio, halogen, heterocyclyl, aryl, heteroaryl, arylalkyl, or O-silyl; and
R⁵ represents 0 to 3 substituents, where each is independently selected from the group consisting of alkyl, cycloalkyl, alkoxy, alkylamino, alkylthio, halogen, heterocyclyl, aryl, heteroaryl, arylalkyl, and O-silyl.

5. The process of claim 3, where the C-nitroso dienophile is a compound of formula (Ic): 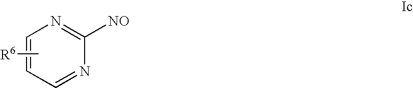
where, R⁶ represents 0 to 3 substituents, independently selected from the group consisting of alkyl, cycloalkyl, alkoxy, alkylamino, alkylthio, halogen, heterocyclyl, aryl, heteroaryl, arylalkyl, and O-silyl.

6. The process of claim 3, where the C-nitroso dienophile is a compound of formula (Id): 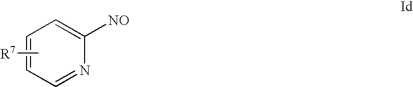
where, R⁷ represents 0 to 4 substituents, each of which is independently selected from the group consisting of alkyl, cycloalkyl, alkoxy, alkylamino, alkylthio, halogen, heterocyclyl, aryl, heteroaryl, arylalkyl, and O-silyl.

7. The process of claim 2, where the C-nitroso dienophile is a compound of formula (Ia): 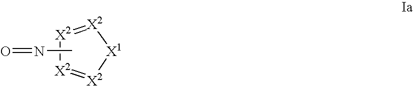
where:
each X¹ is selected from the group consisting of —NR²_, —O—, and —S—;
R² is independently selected from the group consisting of hydrogen, alkyl, cycloalkyl, alkoxy, alkylamino, alkylthio, halogen, heterocyclyl, aryl, heteroaryl, arylalkyl, and O-silyl;
each X² is independently selected from the group consisting of —CR³— and —N—;
R³ is independently selected from the group consisting of hydrogen, alkyl, cycloalkyl, alkoxy, alkylamino, alkylthio, halogen, heterocyclyl, aryl, heteroaryl, arylalkyl, and O-silyl.

8. The process of claim 7, where the C-nitroso dienophile is a compound of formula (Ie): 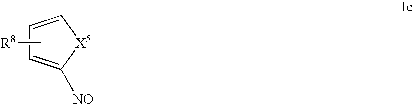
where:
R⁸ represents 0 to 3 substituents, each of which is independently selected from the group consisting of alkyl, cycloalkyl, alkoxy, alkylamino, alkylthio, halogen, heterocyclyl, aryl, heteroaryl, arylalkyl, and O-silyl;
X⁵ is selected from the group consisting of —NR⁹—, —O—, and —S—;
R⁹ is selected from the group consisting of hydrogen, alkyl, cycloalkyl, alkoxy, alkylamino, alkylthio, halogen, heterocyclyl, aryl, heteroaryl, arylalkyl, and O-silyl

9. The process of claim 1, where the diene is a compound of formula (II): 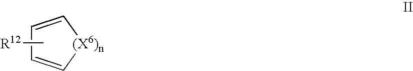
where,
each X⁶ is independently selected from the group consisting of —CR⁹R¹⁰—, —NR¹—, —O—, and —S—;
R⁹, R¹⁰, R¹¹ are each independently selected from the group consisting of hydrogen, alkyl, cycloalkyl, alkoxy, alkylamino, alkylthio, halogen, heterocyclyl, aryl, heteroaryl, arylalkyl, and O-silyl;
n is 1, 2, 3, or 4; and
R¹² represents 0 to 4 substituents, each of which is independently selected from the group consisting of alkyl, cycloalkyl, alkoxy, alkylamino, alkylthio, halogen, heterocyclyl, aryl, heteroaryl, arylalkyl, and O-silyl.

10. The process of claim 1, where the diene is selected from the following formulae (IIa, IIb, IIc, and IId): 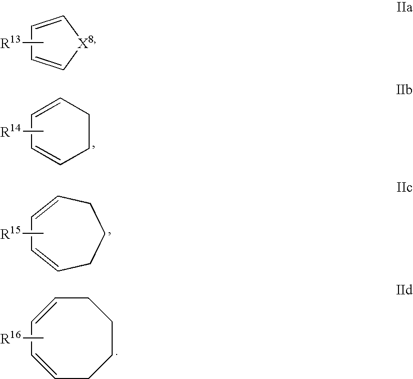
where,
R¹³ represents 0 to 4 substituents with respect to IIa;
R¹⁴ represents 0 to 6 substituents with respect to IIb;
R¹⁵ represents 0 to 10 substituents with respect to IIc;
R¹⁶ represents 0 to 12 substituents with respect to IId;
R¹³, R¹⁴, R¹⁵, and R¹⁶ are each independently selected from the group consisting of alkyl, cycloalkyl, alkoxy, alkylamino, alkylthio, halogen, heterocyclyl, aryl, heteroaryl, arylalkyl, and O-silyl;
X⁸ is selected from the group consisting of —CR¹⁷R¹⁸—, —NR¹⁹—, —O—, and —S—; and
R¹⁷, R¹⁸, and R¹⁹ are each independently selected from the group consisting of alkyl, cycloalkyl, alkoxy, alkylamino, alkylthio, halogen, heterocyclyl, aryl, heteroaryl, arylalkyl, and O-silyl.

11. The process of claim 1, where the diene is a compound of formula (IIe): 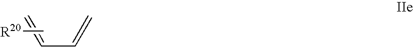
where, R²⁰ represents 0 to 6 substituents, each of which is independently selected from the group consisting of alkyl, cycloalkyl, alkoxy, alkylamino, alkylthio, halogen, heterocyclyl, aryl, heteroaryl, arylalkyl, and O-silyl.

12. The process of claim 1, where the diene is an unsubstituted or substituted compound selected from the following formulae:

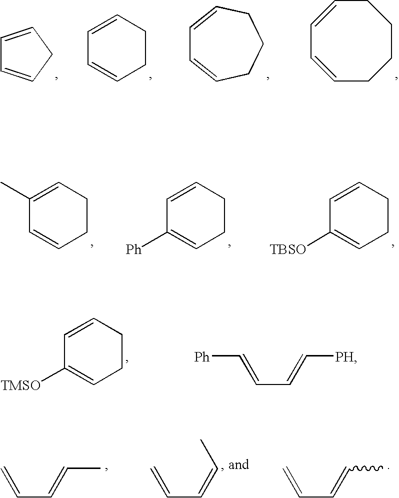

13. The process of claim 1, where the metal is a Lewis acid.

14. The process of claim 1, where the asymmetric bidentate ligand is C-2 symmetric.

15. The process of claim 1, where the asymmetric bidentate ligand is a compound of formula (III): 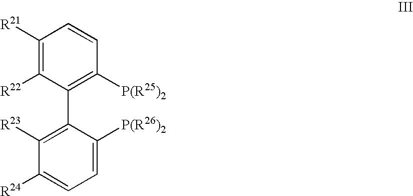
where:
R²¹, R²², R²³, and R²⁴ are each independently selected from the group consisting of hydrogen, alkyl, cycloalkyl, alkoxy, halogen, heterocyclyl, aryl, heteroaryl, arylalkyl, and O-silyl;
R²⁵ and R²⁶ are each independently selected from the group consisting of alkyl, cycloalkyl, heterocyclyl, aryl, arylalkyl, and heteroaryl.

16. The process of claim 1, where the asymmetric bidentate ligand is an unsubstituted or substituted group selected from the following formulae:

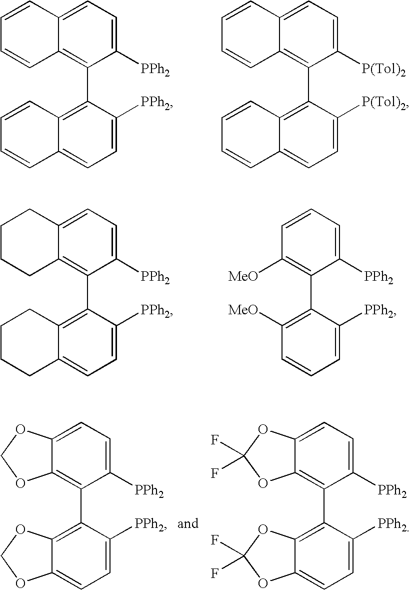

17. The process of claim 1, where the metal and the asymmetric bidentate ligand (IV) form a complex.

18. The process of claim 17, where the ratio of asymmetric bidentate ligand to metal is about 1.0 to about 1.0.

19. The process of claim 1, where the quantity of the asymmetric bidentate ligand and metal complex is about 0.05 to about 0.25 equivalents.

20. The process of claim 1, where the reacting step is performed in solvent selected from the group consisting of methylene chloride, chloroform, tetrahydrofuran, benzene, toluene, and acetonitrile.

21. The process of claim 1, where the reacting step is performed at about −85° C. to about 20° C.

22. The process of claim 1, where the reacting step is performed under inert gas.

23. The process of claim 1, where the ratio of the C-nitroso dienophile to the diene is about 1.0 to about 1.5.

24. The process of claim 3, where the Diels-Alder reaction is performed with diene II, and provides cycloadduct (IV):

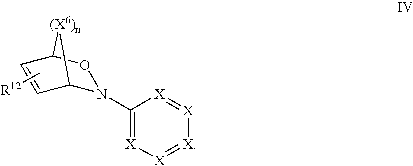

25. The process of claim 7, where the Diels-Alder reaction is performed with diene II, and provides cycloadduct (IVa):

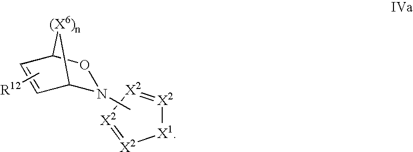

26. The process of claim 1 further comprising the step of cleaving the nitrogen-oxygen bond of the dihydro-1,2-oxazine cycloadduct.

27. The process of claim 26, where the cleaving step is performed using Mo(CO)₆, NaBH₄, and aqueous MeCN.

28. The process of claim 2, where the substrate is a C-nitroso compound of formula (If): 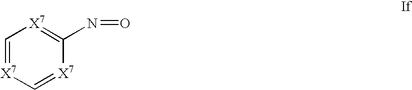
where,
each X⁷ is independently selected from the group consisting of —CR²⁷— and —N—; and
at least one X⁷ is —N—; and
R²⁷ is independently selected from the group consisting of hydrogen, alkyl, cycloalkyl, alkoxy, halogen, heterocyclyl, aryl, heteroaryl, arylalkyl, and O-silyl.

29. The process of claim 28, where the bond between the nitrogen of the nitroso group and the carbon of the aromatic ring is cleaved.

30. The process of claim 1, where the diene is selected from the following formulae: 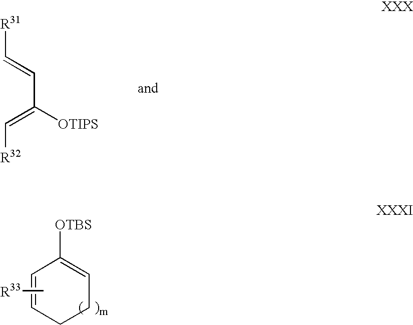
where,
R³¹ and R³² are each independently selected from the group consisting of alkyl, cycloalkyl, alkoxy, alkylamino, alkylthio, aryl, arylalkyl, heterocyclyl, heteroaryl, halogen, silyloxy, carboxylic acid, ester, alkene, azide, amine, hydroxyl, imine, ketone, thiole, amide, silyl, nitrile, sulfoxide, sulfone, sulfonamide and nitroso;
R³³ represents 0 to 4 substituents each of which is independently selected from the group consisting of alkyl, cycloalkyl, alkoxy, alkylamino, alkylthio, aryl, arylalkyl, heterocyclyl, heteroaryl, halogen, silyloxy, carboxylic acid, ester, alkene, azide, amine, hydroxyl, imine, ketone, thiole, amide, silyl, nitrile, sulfoxide, sulfone, sulfonamide and nitroso; and
m is 0, 1 or 2.
